SEQUENCE LISTING (4 sequences)

SEQ ID NO: 1 - DNA, 8475 bp, synthetic construct
  source 1..8475: mol_type = other DNA
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatcgctagcgctaccggactcagatctcgagctcaagcttcgaattcgccgccaccatggctctgcccgtcaccgctctgctgctgcctctggctctgctgctgcacgctgctcgccctcaggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagcggaggaggaggatctgaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagatccgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcacacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 2 - DNA, 5779 bp, synthetic construct
  source 1..5779: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcacacatttccccgaaaagtgccacctgacgt

SEQ ID NO: 3 - DNA, 8846 bp, synthetic construct
  source 1..8846: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcacacatttccccgaaaagtgccacctgacgt

SEQ ID NO: 4 - DNA, 4041 bp, synthetic construct
  source 1..4041: mol_type = other DNA
gtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

(19) United States
(12) Patent Application Publication
(10) Pub. No.: US 2025/0213690 A1
(43) Pub. Date: Jul. 3, 2025

(54) METHODS IN LENTIVIRAL MANUFACTURING FOR PRODUCTION OF CAR-T CELL DRUG PRODUCTS

(71) Applicant: Janssen Biotech, Inc., Horsham, PA (US)

(72) Inventors: Ravinder BHATIA, Blue Bell, PA (US); Nafiseh POORNEJAD, North Wales, PA (US)

(21) Appl. No.: 18/906,622

(22) Filed: Oct. 4, 2024

Related U.S. Application Data
(60) Provisional application No. 63/588,148, filed on Oct. 5, 2023.

(51) Int. Cl. A61K 40/31; A61K 40/11; C12N 15/86

(52) U.S. Cl. CPC A61K 40/31; A61K 40/11; C12N 15/86; C12N 2740/15043; C12N 2740/15052

(57) ABSTRACT

The present application relates to improvements in lentiviral manufacturing for producing a CAR-T cell drug product, wherein the manufacturing method comprises changes to the time between host cell transfection and harvest, and new vector ratios for transfection. Presented herein are methods of preparing lentivirus with various vector ratios and times between host cell transfection and harvest, as well as transfection composition comprising the various vector ratios.

### CROSS-REFERENCE TO RELATED APPLICATIONS

This application claims the benefit of U.S. Provisional Application No. 63/588,148 filed on Oct. 5, 2023, which is hereby incorporated by reference in its entirety.

### SEQUENCE LISTING

The instant application contains a Sequence Listing which has been submitted electronically in XML format and is hereby incorporated by reference in its entirety. Said XML copy, created on Aug. 28, 2024, is named “258199091502 (JBI6821WOPCT1) Sequence Listing.xml” and is 31,678 bytes in size.

### FIELD

The present application relates to improvements in lentiviral manufacturing for CAR-T cell preparation.

### BACKGROUND

Chimeric antigen receptor T-cell (CAR-T) therapy utilizes isolated T cells that have been genetically modified to enhance their specificity for a specific tumor associated antigen. These T cells are typically autologous, where the T cells are isolated from the patient who will receive the T cell therapy. These isolated T cells are then genetically modified using lentiviruses to express a chimeric antigen receptor (CAR). T cells expressing chimeric antigen receptors (CAR-T cells) can induce tumor immunoreactivity.

Lentiviruses prepared for genetic modification of T cells, using the vector packaging systems that are currently available in the prior art, have a low concentration of viable viruses and a low infectious titer. To achieve an ideal effect of T cell transduction, it is necessary to add a much higher dose of lentiviruses with the disadvantages of higher costs, excess residual substances and poor safety performance. Although four-vector packaging systems have also been used in the prior art to replace the three-vector packaging system, the proper ratio at which the vectors can be combined to provide higher infectious titer while ensuring good safety performance has not been determined yet. Thus, there remains an urgent need to screen a suitable ratio of the four vectors for packaging lentiviruses to genetically modify isolated T cells for CAR-T therapy.

### SUMMARY

Provided herein are methods of preparing a lentivirus for the manufacture of a chimeric antigen receptor (CAR) T-cell (CAR-T) drug product, the method comprising: transfecting host cells with a transfection composition, wherein the transfection composition comprises: a first vector comprising a polynucleotide encoding a CAR, a second vector, a third vector, and a fourth vector, and a transfection media; wherein the ratio of the first vector to the second vector to the third vector to the fourth vector is 2:1:1:1, 11:3:1:5, or 12:2:1:5, culturing the transfected host cells to proliferate, and harvesting the lentivirus, wherein the harvesting occurs about 24-hours after transfection.

Also provided herein are methods of preparing a lentivirus for the manufacture of a chimeric antigen receptor (CAR) T-cell (CAR-T) drug product, the method comprising: transfecting host cells with a transfection composition, wherein the transfection composition comprises: a first vector comprising a polynucleotide encoding a CAR, a second vector, a third vector, and a fourth vector, and a transfection media; wherein the ratio of the first vector to the second vector to the third vector to the fourth vectors is 11:3:1:5 or 12:2:1:5, culturing the transfected host cells to proliferate, and harvesting the lentivirus, wherein the harvesting occurs less than or equal to about 48-hours after transfection.

In some embodiments, the ratio is 2:1:1:1. In some embodiments, the ratio is 11:3:1:5. In some embodiments, the ratio is 12:2:1:5. In some embodiments, the harvesting occurs about 24-hours after transfection. In some embodiments, the first vector, second vector, third vector, and fourth vector comprise lentiviral vectors. In some embodiments, the ratio is 2:1:1:1 and the harvesting occurs about 24-hours after transfection. In some embodiments, the ratio is 11:3:1:5 and the harvesting occurs about 24-hours after transfection. In some embodiments, the ratio is 12:2:1:5 and the harvesting occurs about 24-hours after transfection.

In some embodiments, the second vector comprises a polynucleotide encoding a lentiviral envelope protein. In some embodiments, the lentiviral envelope protein is vesicular stomatitis virus G (VSVG). In some embodiments, the third vector comprises a polynucleotide encoding GAG and POL. In some embodiments, the fourth vector comprises a polynucleotide encoding REV.

In some embodiments, the culturing occurs in a bioreactor. In some embodiments, the bioreactor is a 2 L, 10 L or 50 L bioreactor. In some embodiments, the host cells are HEK 293 cells. In some embodiments, the host cells are HEK 293F cells. In some embodiments, the CAR immunospecifically targets B-cell maturation antigen (BCMA).

Provided are methods of preparing a lentivirus for the manufacture of a BCMA CAR-T drug product, the method comprising: transfecting host cells with a transfection composition, wherein the transfection composition comprises: a first vector comprising a polynucleotide encoding a CAR that immunospecifically targets BCMA, a second vector, a third vector, and a fourth vector, and a transfection media; wherein the ratio of the first vector to the second vector to the third vector to the fourth vector is 2:1:1:1, 11:3:1:5, or 12:2:1:5, culturing the transfected host cells to proliferate, and harvesting the lentivirus, wherein the harvesting occurs about 24-hours after transfection.

Also provided herein are methods of preparing a lentivirus for the manufacture of a BCMA CAR-T drug product, the method comprising: transfecting host cells with a transfection composition, wherein the transfection composition comprises: a first vector comprising a polynucleotide encoding a CAR that immunospecifically targets BCMA, a second vector, a third vector, and a fourth vector, and a transfection media; wherein the ratio of the first vector to the second vector to the third vector to the fourth vector is 11:3:1:5 or 12:2:1:5, culturing the transfected host cells to proliferate, and harvesting the lentivirus, wherein the harvesting occurs less than or equal to about 48-hours after transfection.

In some embodiments, the ratio is 2:1:1:1. In some embodiments, ratio is 11:3:1:5. In some embodiments, the ratio is 12:2:1:5. In some embodiments, the harvesting occurs about 24-hours after transfection. In some embodiments, the first vector, second vector, third vector, and fourth vector comprise lentiviral vectors. In some embodiments, the harvesting occurs about 24-hours after transfection. In some embodiments, the harvesting occurs about 24-hours after transfection. In some embodiments, the ratio is 12:2:1:5 and the harvesting occurs about 24-hours after transfection.

In some embodiments, the second vector comprises a polynucleotide encoding a lentiviral envelope protein. In some embodiments, the lentiviral envelope protein is vesicular stomatitis virus G (VSVG). In some embodiments, the third vector comprises a polynucleotide encoding GAG and POL. In some embodiments, the fourth vector comprises a polynucleotide encoding REV.

In some embodiments, the culturing occurs in a bioreactor. In some embodiments, the bioreactor is a 2 L, 10 L or 50 L bioreactor. In some embodiments, the host cells are HEK 293 cells. In some embodiments, the host cells are HEK 293F cells.

In some embodiments, the first vector comprises SEQ ID NO: 1. In some embodiments, second vector comprises SEQ ID NO: 2. In some embodiments, the third vector comprises SEQ ID NO: 3. In some embodiments, the fourth vector comprises SEQ ID NO: 4. In some embodiments, the CAR-T drug product is ciltacabtagene autolucel (cilta-cel).

Provided herein is a transfection composition, comprising: a first vector comprising a polynucleotide encoding a CAR, a second vector, a third vector, and a fourth vector, and a transfection media; wherein the ratio of the first vector to the second vector to the third vector to the fourth vector is 2:1:1:1, 11:3:1:5, or 12:2:1:5.

In some embodiments, the ratio is 2:1:1:1. In some embodiments, the ratio is 11:3:1:5. In some embodiments, the ratio is 12:2:1:5.

In some embodiments, the second vector comprises a polynucleotide encoding a lentiviral envelope protein. In some embodiments, the lentiviral envelope protein is vesicular stomatitis virus G (VSVG). In some embodiments, the third vector comprises a polynucleotide encoding GAG and POL. In some embodiments, the fourth vector comprises a polynucleotide encoding REV.

In some embodiments, the CAR immunospecifically targets B-cell maturation antigen (BCMA). In some embodiments, the first vector comprises SEQ ID NO: 1. In some embodiments, second vector comprises SEQ ID NO: 2. In some embodiments, the third vector comprises SEQ ID NO: 3. In some embodiments, the fourth vector comprises SEQ ID NO: 4.

Provided here in is a method of manufacturing a BCMA CAR-T drug product, the method comprising: providing activated T cells from apheresis material from a subject having multiple myeloma, contacting the T cells with lentivirus prepared by any method disclosed herein or by the use of the transfection composition disclosed herein, culturing the transfected T cells to proliferate, and harvesting the CAR-T drug product. In some embodiments, the BCMA CAR-T drug product is ciltacabtagene autolucel (cilta-cel). Also provided herein is a method of treating multiple myeloma, the method comprising administrating a CAR-T drug product prepared by any method disclosed herein to a subject in need thereof.

### BRIEF DESCRIPTION OF THE DRAWINGS

FIG. 1 is a flow diagram showing the steps in a CAR T cell lentiviral manufacturing process.

FIGS. 2A-2D show vector maps used in a four-vector lentiviral manufacturing process. FIG. 2A depicts a transfer vector contains a gene of interest (“GOI”), here a CAR coding sequence. FIG. 2B depicts an envelope vector encoding VSVG (“MD2g”). FIG. 2C depicts a first packaging vector encoding GAG and POL (“MDLg”). FIG. 2D depicts a second packaging vector encoding REV (“RSV-REV”).

FIGS. 3A and 3B are graphs showing the individual ratio profiles of each of the four vectors (GOI, MD2G, MDLg, and RSV-REV) involved in the CAR T lentiviral manufacturing process. FIG. 3A shows infectious titer by SUPT1, and FIG. 3B shows infectious titer by AD TU Flow.

FIGS. 4A and 4B are graphs showing the individual ratio profiles of each of the four vectors (GOI, MD2G, MDLg, and RSV-REV) involved in the CAR T lentiviral manufacturing process, with the MDLg and RSV-REV vector controlled at a fixed ratio. FIG. 4A shows infectious titer by SUPT1, and FIG. 4B shows infectious titer by AD TU Flow.

FIGS. 5A and 5B depict individual value plots of SUPT1 (FIG. 5A) and API P24 (FIG. 5B) for a control 2:1:1:1 vector ratio (SOP), an 11:3:1:5 vector ratio (Vector Ratio 1) and a 12:2:1:5 vector ratio (Vector Ratio 2).

FIGS. 6A-6D depict value plots of SUPT1 (FIG. 6A), P24 (FIG. 6B), HCP (FIG. 6C), and HCDNA (FIG. 6D) for 24-, 36-, 48-, and 72-hour production conditions.

FIGS. 7A-7D depict the differences between four manufacturing conditions: a 24 hour harvest time with a 2:1:1:1 vector ratio (n=23), a 24 hour harvest time with a 11:3:1:5 vector ratio (n=26), a 48 hour harvest time with a 2:1:1:1 vector ratio (n=25), or a 48 hour harvest time with a 11:3:1:5 vector ratio (n=19). FIG. 7A is a graph of the infectious titers between the groups, while FIGS. 7B-7D graph various impurities (infectious particles, FIG. 7B; host cell DNA, FIG. 7C; host cell protein, FIG. 7D).

FIGS. 8A-8D depict the upstream results at harvest of a 48-hour post transfection harvest time with a 2:1:1:1 vector ratio (control), a 24-hour post transfection harvest time with a 2:1:1:1 vector ratio, and a 24-hour post transfection harvest time with a 11:3:1:5 vector ratio. FIG. 8A shows IT flow, FIG. 8B shows P24 levels, FIG. 8C host cell protein levels, and FIG. 8D shows host cell DNA levels.

FIGS. 9A-9G depict the downstream results post-production processing and formulation analysis. FIG. 9A shows TU-flow titer, FIG. 9B shows IT-PCR titer, FIG. 9C shows p24 levels, FIG. 9D shows P/I levels, FIG. 9E shows host cell protein levels, FIG. 9F shows host cell DNA levels, and FIG. 9G shows vector DNA levels.

FIGS. 10A-10D depict the final drug product results for a 48-hour post transfection harvest time with a 2:1:1:1 vector ratio (Control), a 24-hour post transfection harvest time with a 2:1:1:1 vector ratio (Option 1), and a 24-hour post transfection harvest time with a 11:3:1:5 vector ratio (Option 2). FIG. 10A shows CAR plus T-cell cell percentage, FIG. 10B shows drug product viability, FIG. 10C shows vector copy number, and FIG. 10D shows drug product potency.

FIG. 11 is a flow chart depicting the process for a full end-to-end drug product manufacturing run starting from a single apheresis sample, but where the run was split into four separate, but simultaneous, transductions and expansions using lentivirus manufactured under different conditions: 1) a 50 L bioreactor, 48-hour post-transfection culture time, 2:1:1:1 vector ratio control group (50 L Control); 2) a 10 L bioreactor, 48-hour post-transfection culture time, 2:1:1:1 vector ratio control group (10 L Control); 3) a 10 L bioreactor, 24-hour post-transfection culture time, 2:1:1:1 vector ratio experimental group (10 L Option 1); and 4) a 10 L bioreactor, 24-hour post-transfection culture time, 11:3:1:5 vector ratio experimental group (10 L Option 2).

FIGS. 12A-12E depict the results from the experiments performed with separate apheresis material from different patients. FIG. 12A shows the CAR T-cell percentage in the drug product, FIG. 12B shows drug product viability, FIG. 12C shows vector integration, FIG. 12D shows impact to various T cell immunophenotypes, and FIG. 12E shows cell growth. In each figure, apheresis material from the first patient is shown in the top graph, and apheresis material from the second patient is shown in the bottom graph.

FIG. 13 depicts a flow diagram illustrating the downstream purification process applied to harvested material following upstream bioreactor operations.

FIGS. 14A-14D depict the upstream results at harvest of a 48-hour post transfection harvest time with a 2:1:1:1 vector ratio (control), a 24-hour post transfection harvest time with a 2:1:1:1 vector ratio, a 24-hour post transfection harvest time with a 11:3:1:5 vector ratio, a 48-hour post transfection harvest time with a 2:1:1:1 vector ratio (control) at the 50 L scale only, and a 24-hour post transfection harvest time with a 2:1:1:1 vector ratio at the 50 L scale only. FIG. 14A shows IT flow, FIG. 14B shows P24 levels, FIG. 14C host cell protein levels, and FIG. 14D shows host cell DNA levels.

FIGS. 15A-15G depict the downstream results post-production processing and formulation analysis with a 2:1:1:1 vector ratio (Control), a 24-hour post transfection harvest time with a 2:1:1:1 vector ratio (Option 1), a 24-hour post transfection harvest time with a 11:3:1:5 vector ratio (Option 2), a 48-hour post transfection harvest time with a 2:1:1:1 vector ratio (control) at the 50 L scale only, and a 24-hour post transfection harvest time with a 2:1:1:1 vector ratio at the 50 L scale only. FIG. 15A shows TU-flow titer, FIG. 15B shows IT-PCR titer, FIG. 15C shows p24 levels, FIG. 15D shows P/I levels, FIG. 15E shows host cell protein levels, FIG. 15F shows host cell DNA levels, and FIG. 15G shows vector DNA levels.

FIGS. 16A-16D depict the final drug product results for a 48-hour post transfection harvest time with a 2:1:1:1 vector ratio (Control), a 24-hour post transfection harvest time with a 2:1:1:1 vector ratio (Option 1), a 24-hour post transfection harvest time with a 11:3:1:5 vector ratio (Option 2), a 48-hour post transfection harvest time with a 2:1:1:1 vector ratio (control) at the 50 L scale only, and a 24-hour post transfection harvest time with a 2:1:1:1 vector ratio at the 50 L scale only. FIG. 16A shows CAR plus T-cell cell percentage, FIG. 16B shows drug product viability, FIG. 16C shows vector copy number, and FIG. 16D shows drug product potency.

FIGS. 17A-17D depict the results from the experiments performed with the method shown in FIGS. 16A-16D but run twice with separate apheresis material from different patients. FIG. 17A shows the CAR T-cell percentage in the drug product, FIG. 17B shows drug product viability, FIG. 17C shows vector integration, FIG. 17D shows drug product potency. In each figure, apheresis material from the first patient is shown in the top graph, and apheresis material from the second patient is shown in the bottom graph.

### DETAILED DESCRIPTION

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

In an attempt to help the reader of the present application, the description has been separated in various paragraphs or sections. These separations are not considered as disconnecting the substance of a paragraph or section from the substance of another paragraph or section. To the contrary, the present description encompasses all the combinations of the various sections, paragraphs and sentences that can be contemplated.

As used herein, the indefinite articles “a”, “an” and “the” should be understood to include plural reference unless the context clearly indicates otherwise.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the system under study, and can be readily appreciated by one of ordinary skill in the art.

A “chimeric antigen receptor” or “CAR” is an artificially constructed hybrid protein or polypeptide containing the antigen binding domains of an antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express an antibody binding domain on its surface, conferring novel antigen specificity that is MHC independent. “BCMA CAR” refers to a CAR having an extracellular binding domain specific for BCMA.

As used herein, the terms “specifically binds”, “specifically recognizes”, or “specific for” refer to measurable and reproducible interactions such as binding between a target and an antigen binding protein (such as a CAR or a VHH domain), which is determinative of the presence of the target in the presence of a heterogeneous population of molecules including biological molecules.

The term “specificity” refers to selective recognition of an antigen binding protein (such as a CAR or a VHH domain) for a particular epitope of an antigen. Natural antibodies, for example, are monospecific.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovine (cows) and swine (pigs) or of the order Perssodactyla, including equines (horses). The mammals may be of the order Primates, Ceboids, or Simoids (monkeys) or of the order Anthropoids (humans and apes). In some embodiments, the mammal is a human.

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques. The disclosure also provides a vector comprising the nucleic acid sequence encoding the CARs disclosed herein. The vector can be, for example, a plasmid, a cosmid, a viral vector (e.g., retroviral or adenoviral), an artificial chromosome (such as a yeast artificial chromosome (YAC), a bacterial artificial chromosome (BAC), or a P1-derived artificial chromosome (PAC)), or a phage. Suitable vectors and methods of vector preparation are well known in the art (see, e.g., Sambrook et al. and Ausubel et al.).

In addition to the nucleic acid sequences encoding the CARs disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CARs disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol, 308: 123-144 (2005)).

In some embodiments, the vector comprises an “enhancer”. The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. Many enhancers from a variety of different sources are well known in the art and are available as or within cloned polynucleotides (e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which can replicate in a host cell and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pB-CMV from Stratagene (La Jolla, CA) represent non-limiting examples of an episomal vector that uses T-antigen and the SV40 origin of replication in lieu of EBNA1 and oriP.

In some embodiments, the vector is an “integrating expression vector,” which may randomly integrate into the host cell's DNA or may include a recombination site to enable recombination between the expression vector and a specific site in the host cell's chromosomal DNA. Such integrating expression vectors may utilize the endogenous expression control sequences of the host cell's chromosomes to effect expression of the desired protein. Examples of vectors that integrate in a site-specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFNI OA (ACT) FLEXI™ from Promega (Madison, WI). In some embodiments, the integrating expression vector may also include components that help its entry into the cells, such as viral particles, liposomes or protein coats, but are not limited to those substances.

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLPl from Life Technologies (Carlsbad, CA)), and retroviral vectors (e.g., the pFB-ERV plus pCFB-EGSH from Stratagene (La Jolla, CA)). In some embodiments, the viral vector is a lentiviral vector. In some embodiments, a virus, such as a lentivirus or an adenovirus, refers to a complete virus, including the viral capsid and the viral vector contained in the viral capsid.

The vector comprising the inventive nucleic acid encoding the CAR can be introduced into a host cell that can express the CAR encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK 293 cells, and the like. In a preferred aspect, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cells are HEK 293F cells. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell. In some embodiments, the host cell is an autologous cell.

For purposes of amplifying or replicating the recombinant expression vector, the host cell may be a prokaryotic cell, e.g., a DH5a cell. For purposes of producing a virus from a viral expression vector, the host cell may be a eukaryotic cell, e.g., a HEK 293 cell. For purposes of producing a recombinant CAR, the host cell can be a mammalian cell. The host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8+ T-cell (e.g., a cytotoxic T-cell), a tumor infiltrating cell, a memory T-cell, a naive T-cell, and the like. In one aspect, the T-cell is a CD8+ T-cell or a CD4+ T-cell. T-cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

In some embodiments, the nucleic acid sequences encoding a CAR may be introduced into a cell by “transfection”, “transformation”, or “transduction”. “Transfection”, “transformation”, or transduction”, as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available

The embodiments herein generally relate to improved methods for manufacturing T cell compositions and drug products, specifically CAR-T cell drug products. Manufacturing of a CAR-T cell drug product is a complex, multi-day process. CAR-T cell manufacturing processes have been described, for example, in Hollyman et al., J. Immunother. 2009, 32:169-180, U.S. Patent Publication 2022/0195060A1, and U.S. Patent Publication 2022/0017862A1, each of which are incorporated by reference in their entirety.

An essential step in manufacturing CAR-T cell drug products is providing a lentivirus to transduce enriched T cells. The transduced T cells then produce and express the CAR protein, creating the CAR-T cells. Manufacturing the lentivirus is itself a complex multi-day process that can be generally divided into upstream processing where the production is set up, and downstream processing where the lentivirus is harvested and prepared.

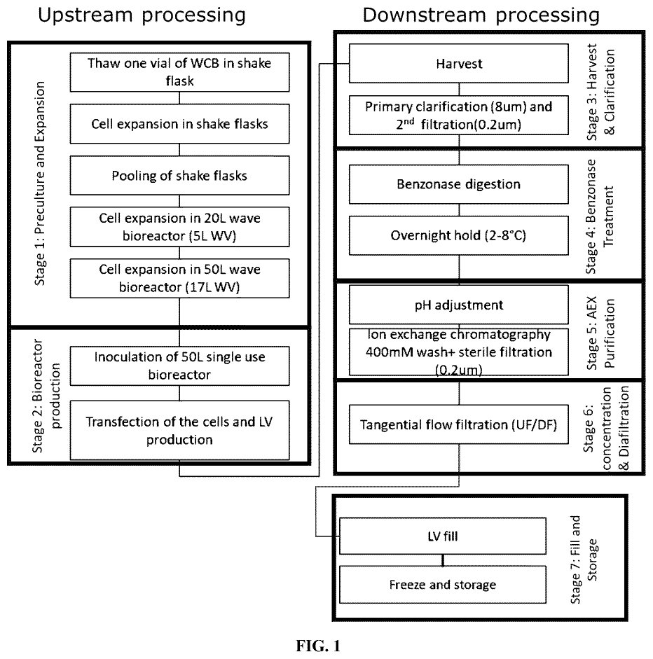

FIG. 1 provides an overview of the lentiviral manufacturing process. Without wishing to be bound by theory, upstream processing can be generally subdivided into two stages: 1) preculture and expansion, which can take up to 22 days, and 2) bioreactor production, which can take up to 4 days. The embodiments herein relate to an improved transfection process that allows for a shorter time frame from transfection to harvest, i.e., the bioreactor production stage, with lower impurities and little to no loss in quality or quantity of the final lentivirus product at harvest. In some embodiments, the method comprises a modified ratio of the vectors used in the transduction process. In some embodiments, the method comprises a shortened time from transduction to harvest. In some embodiments, the method comprises both a modified vector ratio and a shortened time from transduction to harvest.

Provided herein are transfection compositions comprising a four-vector lentiviral system, methods of preparing a lentivirus for the manufacture of a chimeric antigen receptor CAR-T drug product, methods of manufacturing a CAR-T drug product, and methods of treating multiple myeloma.

### Transfection Compositions

Provided herein are transfection compositions comprising a four-vector system. In some embodiments, the four-vector system comprises a first vector, a second vector, a third vector, a fourth vector, and a transfection media.

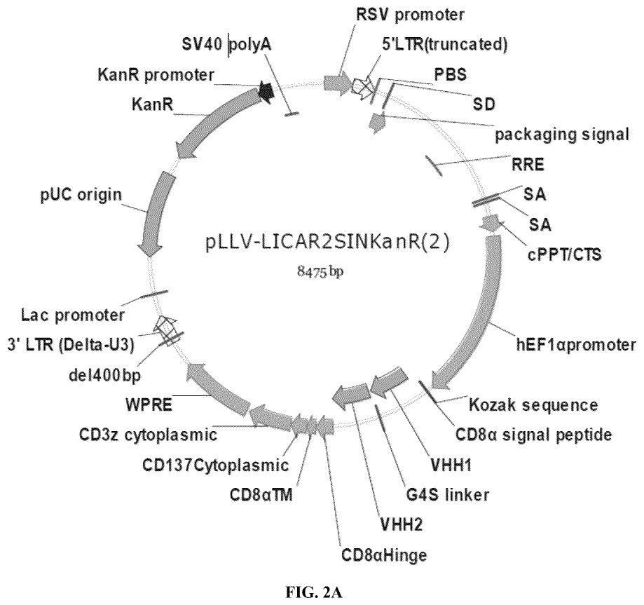

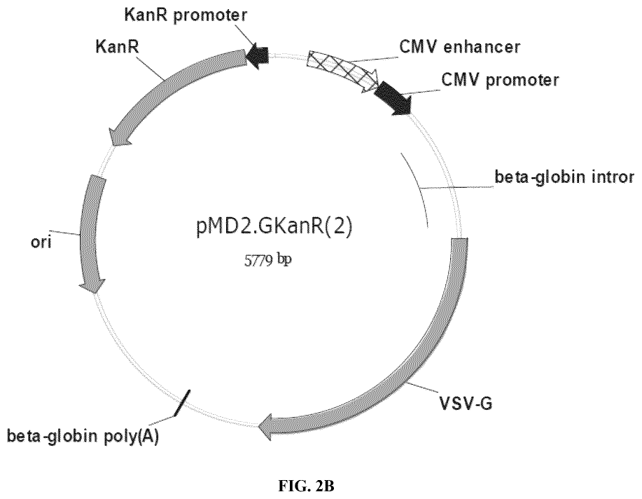

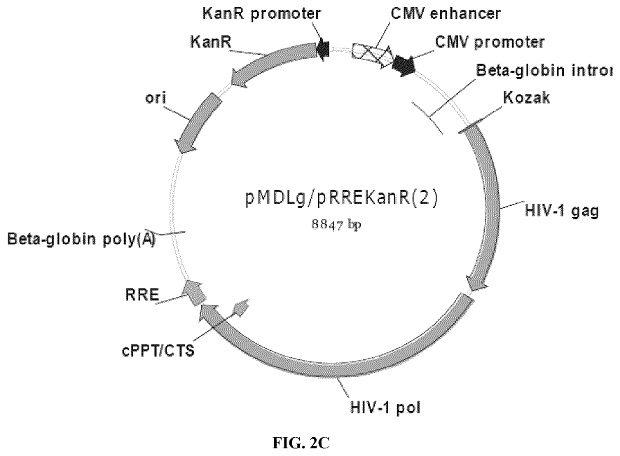

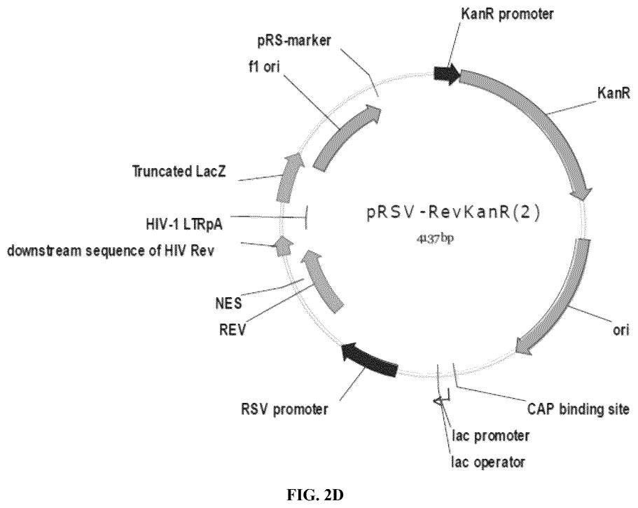

In some embodiments, the first vector comprises a polynucleotide encoding a gene of interest (GOI). In some embodiments, a polynucleotide encoding a chimeric antigen receptor (CAR). In some embodiments, the first vector comprises a nucleic acid sequence that is at least 90%, at least 91%, at least 92%, at least 93%, at least 94%, at least 95%, at least 96%, at least 97%, at least 98%, or at least 99% identical to the following nucleic acid sequence: tgtagtcttatgcaatactcttgtagtcttgcaacatggtaacgatgagttagcaacatgccttacaaggagagaaaaagcaccgtgcatg ccgattggtggaagtaaggtggtacgatcgtgccttattaggaaggcaacagacgggtctgacatggattggacgaaccactgaattg ccgcattgcagagatattgtatttaagtgcctagctcgatacataaacgggtctctctggttagaccagatctgagcctgggagctctctg gctaactagggaacccactgcttaagcctcaataaagcttgccttgagtgcttcaagtagtgtgtgcccgtctgttgtgtgactctggtaa ctagagatccctcagacccttttagtcagtgtggaaaatctctagcagtggcgcccgaacagggacttgaaagcgaaagggaaacca gaggagctctctcgacgcaggactcggcttgctgaagcgcgcacggcaagaggcgaggggcggcgactggtgagtacgccaaaa attttgactagcggaggctagaaggagagagatgggtgcgagagcgtcagtattaagcgggggagaattagatcgcgatgggaaaa aattcggttaaggccagggggaaagaaaaaatataaattaaaacatatagtatgggcaagcagggagctagaacgattcgcagttaat cctggcctgttagaaacatcagaaggctgtagacaaatactgggacagctacaaccatcccttcagacaggatcagaagaacttagat cattatataatacagtagcaaccctctattgtgtgcatcaaaggatagagataaaagacaccaaggaagctttagacaagatagaggaa gagcaaaacaaaagtaagaccaccgcacagcaagcggccggccgctgatcttcagacctggaggaggagatatgagggacaattg gagaagtgaattatataaatataaagtagtaaaaattgaaccattaggagtagcacccaccaaggcaaagagaagagtggtgcagag agaaaaaagagcagtgggaataggagctttgttccttgggttcttgggagcagcaggaagcactatgggcgcagcgtcaatgacgct gacggtacaggccagacaattattgtctggtatagtgcagcagcagaacaatttgctgagggctattgaggcgcaacagcatctgttgc aactcacagtctggggcatcaagcagctccaggcaagaatcctggctgtggaaagatacctaaaggatcaacagctcctggggatttg gggttgctctggaaaactcatttgcaccactgctgtgccttggaatgctagttggagtaataaatctctggaacagatttggaatcacacg acctggatggagtgggacagagaaattaacaattacacaagcttaatacactccttaattgaagaatcgcaaaaccagcaagaaaaga atgaacaagaattattggaattagataaatgggcaagtttgtggaattggtttaacataacaaattggctgtggtatataaaattattcataat gatagtaggaggcttggtaggtttaagaatagtttttgctgtactttctatagtgaatagagttaggcagggatattcaccattatcgtttcag acccacctcccaaccccgaggggacccgacaggcccgaaggaatagaagaagaaggtggagagagagacagagacagatccat tcgattagtgaacggatctcgacggtatcgcctttaaaagaaaaggggggattggggggtacagtgcaggggaaagaatagtagaca taatagcaacagacatacaaactaaagaattacaaaaacaaattacaaaaattcaaaattttcgggtttattacagggacagcagagatc cagtttatcgatgagtaattcatacaaaaggactcgcccctgccttggggaatcccagggaccgtcgttaaactcccactaacgtagaa cccagagatcgctgcgttcccgccccctcacccgcccgctctcgtcatcactgaggtggagaagagcatgcgtgaggctccggtgcc cgtcagtgggcagagcgcacatcgcccacagtccccgagaagttggggggaggggtcggcaattgaaccggtgcctagagaaggt ggcgcggggtaaactgggaaagtgatgtcgtgtactggctccgcctttttcccgagggtgggggagaaccgtatataagtgcagtagt cgccgtgaacgttctttttcgcaacgggtttgccgccagaacacaggtaagtgccgtgtgtggttcccgcgggcctggcctctttacgg gttatggcccttgcgtgccttgaattacttccacgcccctggctgcagtacgtgattcttgatcccgagcttcgggttggaagtgggtggg agagttcgaggccttgcgcttaaggagccccttcgcctcgtgcttgagttgaggcctggcttgggcgctggggccgccgcgtgcgaat ctggtggcaccttcgcgcctgtctcgctgctttcgataagtctctagccatttaaaatttttgatgacctgctgcgacgctttttttctggcaa gatagtcttgtaaatgcgggccaagatctgcacactggtatttcggtttttggggccgcgggcggcgacggggcccgtgcgtcccagc gcacatgttcggcgaggcggggcctgcgagcgcggccaccgagaatcggacgggggtagtctcaagctggccggcctgctctggt gcctggcctcgcgccgccgtgtatcgccccgccctgggcggcaaggctggcccggtcggcaccagttgcgtgagcggaaagatgg ccgcttcccggccctgctgcagggagctcaaaatggaggacgcggcgctcgggagagcgggcgggtgagtcacccacacaaagg aaaagggcctttccgtcctcagccgtcgcttcatgtgactccacggagtaccgggcgccgtccaggcacctcgattagttctcgagcttt tggagtacgtcgtctttaggttggggggaggggttttatgcgatggagtttccccacactgagtgggtggagactgaagttaggccagc ttggcacttgatgtaattctccttggaatttgccctttttgagtttggatcttggttcattctcaagcctcagacagtggttcaaagtttttttcttc catttcaggtgtcgtgaggatcgctagcgctaccggactcagatctcgagctcaagcttcgaattcgccgccaccatggctctgcccgt caccgctctgctgctgcctctggctctgctgctgcacgctgctcgccctcaggtcaaactggaagaatctggcggaggcctggtgcag gcaggacggagcctgcgcctgagctgcgcagcatccgagcacaccttcagctcccacgtgatgggctggtttcggcaggccccag gcaaggagagagagagcgtggccgtgatcggctggagggacatctccacatcttacgccgattccgtgaagggccggttcaccatc agccgggacaacgccaagaagacactgtatctgcagatgaacagcctgaagcccgaggacaccgccgtgtactattgcgcagcaa ggagaatcgacgcagcagactttgattcctggggccagggcacccaggtgacagtgtctagcggaggaggaggatctgaggtgca gctggtggagagcggaggcggcctggtgcaggccggaggctctctgaggctgagctgtgcagcatccggaagaaccttcacaatg ggctggtttaggcaggcaccaggaaaggagagggagttcgtggcagcaatcagcctgtcccctaccctggcctactatgccgagag cgtgaagggcaggtttaccatctcccgcgataacgccaagaatacagtggtgctgcagatgaactccctgaaacctgaggacacagc cctgtactattgtgccgccgatcggaagagcgtgatgagcattagaccagactattgggggcagggaacacaggtgaccgtgagca gcactagtaccacgacgccagcgccgcgaccaccaacaccggcgcccaccatcgcgtcgcagcccctgtccctgcgcccagagg cgtgccggccagcggcggggggcgcagtgcacacgagggggctggacttcgcctgtgatatctacatctgggcgcccttggccgg gacttgtggggtccttctcctgtcactggttatcaccctttactgcaaacggggcagaaagaaactcctgtatatattcaaacaaccatttat gagaccagtacaaactactcaagaggaagatggctgtagctgccgatttccagaagaagaagaaggaggatgtgaactgagagtga agttcagcaggagcgcagacgcccccgcgtaccagcagggccagaaccagctctataacgagctcaatctaggacgaagagagg agtacgatgttttggacaagagacgtggccgggaccctgagatggggggaaagccgagaaggaagaaccctcaggaaggcctgta caatgaactgcagaaagataagatggcggaggcctacagtgagattgggatgaaaggcgagcgccggaggggcaaggggcacg atggcctttaccagggtctcagtacagccaccaaggacacctacgacgcccttcacatgcaggccctgccccctcgctaatctagatc cgcgtctggaacaatcaacctctggattacaaaatttgtgaaagattgactggtattcttaactatgttgctccttttacgctatgtggatacg ctgctttaatgcctttgtatcatgctattgcttcccgtatggctttcattttctcctccttgtataaatcctggttgctgtctctttatgaggagttgt ggcccgttgtcaggcaacgtggcgtggtgtgcactgtgtttgctgacgcaacccccactggttggggcattgccaccacctgtcagctc ctttccgggactttcgctttccccctccctattgccacggcggaactcatcgccgcctgccttgcccgctgctggacaggggctcggct gttgggcactgacaattccgtggtgttgtcggggaagctgacgtcctttccatggctgctcgcctgtgttgccacctggattctgcgcgg gacgtccttctgctacgtcccttcggccctcaatccagcggaccttccttcccgcggcctgctgccggctctgcggcctcttccgcgtctt cgccttcgccctcagacgagtcggatctccctttgggccgcctccccgcctggaattaattctgcagtcgagacctagaaaaacatgga gcaatcacaagtagcaatacagcagctaccaatgctgattgtgcctggctagaagcacaagaggaggaggaggtgggttttccagtc acacctcaggtacctttaagaccaatgacttacaaggcagctgtagatcttagccactttttaaaagaaaagaggggactggaagggct aattcactcccaacgaagacaagatctgctttttgcttgtactgggtctctctggttagaccagatctgagcctgggagctctctggctaac tagggaacccactgcttaagcctcaataaagcttgccttgagtgcttcaagtagtgtgtgcccgtctgttgtgtgactctggtaactagag atccctcagacccttttagtcagtgtggaaaatctctagcagtagtagttcatgtcatcttattattcagtatttataacttgcaaagaaatgaa tatcagagagtgagaggccttgacattgctagcgttttaccgtcgacctctagctagagcttggcgtaatcatggtcatagctgtttcctgt gtgaaattgttatccgctcacaattccacacaacatacgagccggaagcataaagtgtaaagcctggggtgcctaatgagtgagctaac tcacattaattgcgttgcgctcactgcccgctttccagtcgggaaacctgtcgtgccagctgcattaatgaatcggccaacgcgcgggg agaggcggtttgcgtattgggcgctcttccgcttcctcgctcactgactcgctgcgctcggtcgttcggctgcggcgagcggtatcagc tcactcaaaggcggtaatacggttatccacagaatcaggggataacgcaggaaagaacatgtgagcaaaaggccagcaaaaggcc aggaaccgtaaaaaggccgcgttgctggcgtttttccataggctccgcccccctgacgagcatcacaaaaatcgacgctcaagtcaga ggtggcgaaacccgacaggactataaagataccaggcgtttccccctggaagctccctcgtgcgctctcctgttccgaccctgccgctt accggatacctgtccgcctttctcccttcgggaagcgtggcgctttctcatagctcacgctgtaggtatctcagttcggtgtaggtcgttcg ctccaagctgggctgtgtgcacgaaccccccgttcagcccgaccgctgcgccttatccggtaactatcgtcttgagtccaacccggtaa gacacgacttatcgccactggcagcagccactggtaacaggattagcagagcgaggtatgtaggcggtgctacagagttcttgaagt ggtggcctaactacggctacactagaagaacagtatttggtatctgcgctctgctgaagccagttaccttcggaaaaagagttggtagct cttgatccggcaaacaaaccaccgctggtagcggtggtttttttgtttgcaagcagcagattacgcgcagaaaaaaaggatctcaagaa gatcctttgatcttttctacggggtctgacgctcagtggaacgaaaactcacgttaagggattttggtcatgagattatcaaaaaggatctt cacctagatccttttaaattaaaaatgaagttttaaatcaatctaaagtatatatgagtaaacttggtctgacagttagaaaaactcatcgag catcaaatgaaactgcaatttattcatatcaggattatcaataccatatttttgaaaaagccgtttctgtaatgaaggagaaaactcaccgag gcagttccataggatggcaagatcctggtatcggtctgcgattccgactcgtccaacatcaatacaacctattaatttcccctcgtcaaaa ataaggttatcaagtgagaaatcaccatgagtgacgactgaatccggtgagaatggcaaaagtttatgcatttctttccagacttgttcaa caggccagccattacgctcgtcatcaaaatcactcgcatcaaccaaaccgttattcattcgtgattgcgcctgagcgagacgaaatacg cgatcgctgttaaaaggacaattacaaacaggaatcgaatgcaaccggcgcaggaacactgccagcgcatcaacaatattttcacctg aatcaggatattcttctaatacctggaatgctgtttttccggggatcgcagtggtgagtaaccatgcatcatcaggagtacggataaaatg cttgatggtcggaagaggcataaattccgtcagccagtttagtctgaccatctcatctgtaacatcattggcaacgctacctttgccatgttt cagaaacaactctggcgcatcgggcttcccatacaagcgatagattgtcgcacctgattgcccgacattatcgcgagcccatttatacc catataaatcagcatccatgttggaatttaatcgcggcctcgacgtttcccgttgaatatggctcataacaccccttgtattactgtttatgta agcagacagttttattgttcatgatgatatatttttatcttgtgcaatgtaacatcagagattttgagacacgggccagagctgcacacatttc cccgaaaagtgccacctgacgtcgacggatcgggagatcaacttgtttattgcagcttataatggttacaaataaagcaatagcatcaca aatttcacaaataaagcatttttttcactgcattctagttgtggtttgtccaaactcatcaatgtatcttatcatgtctggatcaactggataact caagctaaccaaaatcatcccaaacttcccaccccataccctattaccactgccaattacctgtggtttcatttactctaaacctgtgattcc tctgaattattttcattttaaagaaattgtatttgttaaatatgtactacaaacttagtagtttttaaagaaattgtatttgttaaatatgtactacaa acttagtagt (SEQ ID NO: 1). In some embodiments the first vector comprises the nucleic acid sequence of SEQ ID NO: 1. In some embodiments, the first vector consists of the nucleic acid sequence of SEQ ID NO: 1. In some embodiments, the first vector is depicted in the vector map shown in FIG. 2A and is referred to herein as the “GOI” vector or the “LLV” vector. In some embodiments, the CAR encoded by the first vector is a CAR immunospecifically targets B-cell maturation antigen (BCMA).

In some embodiments, the second vector comprises a polynucleotide encoding a lentiviral envelope protein. In some embodiments, the lentiviral envelope protein is vesicular stomatitis virus G (VSVG). In some embodiments, a VSVG-envelope-pseudotyped lentiviral vector extends the target cell tropism range of the vector and increases the stability of the lentiviral vector, thereby allowing the lentivirus to be concentrated by high-speed centrifugation, and further resulting in higher titer. In some embodiments, the second vector comprises a nucleic acid sequence that is at least 90%, at least 91%, at least 92%, at least 93%, at least 94%, at least 95%, at least 96%, at least 97%, at least 98%, or at least 99% identical to the following nucleic acid sequence: ggatcccctgagggggcccccatgggctagaggatccggcctcggcctctgcataaataaaaaaaattagtcagccatgagcttggc ccattgcatacgttgtatccatatcataatatgtacatttatattggctcatgtccaacattaccgccatgttgacattgattattgactagttatt aatagtaatcaattacggggtcattagttcatagcccatatatggagttccgcgttacataacttacggtaaatggcccgcctggctgacc gcccaacgacccccgcccattgacgtcaataatgacgtatgttcccatagtaacgccaatagggactttccattgacgtcaatgggtgg agtatttacggtaaactgcccacttggcagtacatcaagtgtatcatatgccaagtacgccccctattgacgtcaatgacggtaaatggc ccgcctggcattatgcccagtacatgaccttatgggactttcctacttggcagtacatctacgtattagtcatcgctattaccatggtgatgc ggttttggcagtacatcaatgggcgtggatagcggtttgactcacggggatttccaagtctccaccccattgacgtcaatgggagtttgtt ttggcaccaaaatcaacgggactttccaaaatgtcgtaacaactccgccccattgacgcaaatgggcggtaggcgtgtacggtggga ggtctatataagcagagctcgtttagtgaaccgtcagatcgcctggagacgccatccacgctgttttgacctccatagaagacaccggg accgatccagcctcccctcgaagcttacatgtggtaccgagctcggatcctgagaacttcagggtgagtctatgggacccttgatgtttt ctttccccttcttttctatggttaagttcatgtcataggaaggggagaagtaacagggtacacatattgaccaaatcagggtaattttgcattt gtaattttaaaaaatgetttettettttaatatacttttttgtttatettatttetaatactttccctaatetetttetttcagggcaataatgatacaatg tatcatgcctctttgcaccattctaaagaataacagtgataatttctgggttaaggcaatagcaatatttctg catataaatatttctgcatata aattgtaactgatgtaagaggtttcatattgctaatagcagctacaatccagctaccattctgcttttattttatggttgggataaggctggatt attctgagtccaagctaggcccttttgctaatcatgttcatacctcttatcttcctcccacagctcctgggcaacgtgctggtctgtgtgctg gcccatcactttggcaaagcacgtgagatctgaattctgacactatgaagtgccttttgtacttagcctttttattcattggggtgaattgcaa gttcaccatagtttttccacacaaccaaaaaggaaactggaaaaatgttccttctaattaccattattgcccgtcaagctcagatttaaattg gcataatgacttaataggcacagccttacaagtcaaaatgcccaagagtcacaaggctattcaagcagacggttggatgtgtcatgcttc caaatgggtcactacttgtgatttccgctggtatggaccgaagtatataacacattccatccgatccttcactccatctgtagaacaatgca aggaaagcattgaacaaacgaaacaaggaacttggctgaatccaggcttccctcctcaaagttgtggatatgcaactgtgacggatgc cgaagcagtgattgtccaggtgactcctcaccatgtgctggttgatgaatacacaggagaatgggttgattcacagttcatcaacggaa aatgcagcaattacatatgccccactgtccataactctacaacctggcattctgactataaggtcaaagggctatgtgattctaacctcattt ccatggacatcaccttcttctcagaggacggagagctatcatccctgggaaaggagggcacagggttcagaagtaactactttgcttat gaaactggaggcaaggcctgcaaaatgcaatactgcaagcattggggagtcagactcccatcaggtgtctggttcgagatggctgata aggatctctttgctgcagccagattccctgaatgcccagaagggtcaagtatctctgctccatctcagacctcagtggatgtaagtctaat tcaggacgttgagaggatcttggattattccctctgccaagaaacctggagcaaaatcagagcgggtcttccaatctctccagtggatct cagctatcttgctcctaaaaacccaggaaccggtcctgctttcaccataatcaatggtaccctaaaatactttgagaccagatacatcaga gtcgatattgctgctccaatcctctcaagaatggtcggaatgatcagtggaactaccacagaaagggaactgtgggatgactgggcac catatgaagacgtggaaattggacccaatggagttctgaggaccagttcaggatataagtttcctttatacatgattggacatggtatgttg gactccgatcttcatcttagctcaaaggctcaggtgttcgaacatcctcacattcaagacgctgcttcgcaacttcctgatgatgagagttt attttttggtgatactgggctatccaaaaatccaatcgagcttgtagaaggttggttcagtagttggaaaagctctattgcctcttttttctttat catagggttaatcattggactattcttggttctccgagttggtatccatctttgcattaaattaaagcacaccaagaaaagacagatttatac agacatagagatgaaccgacttggaaagtaactcaaatcctgcacaacagattcttcatgtttggaccaaatcaacttgtgataccatgct caaagaggcctcaattatatttgagtttttaatttttatgaaaaaaaaaaaaaaaaacggaattcaccccaccagtgcaggctgcctatca gaaagtggtggctggtgtggctaatgccctggcccacaagtatcactaagctcgctttcttgctgtccaatttctattaaaggttcctttgtt ccctaagtccaactactaaactgggggatattatgaagggccttgagcatctggattctgcctaataaaaaacatttattttcattgcaatga tgtatttaaattatttctgaatattttactaaaaagggaatgtgggaggtcagtgcatttaaaacataaagaaatgaagagctagttcaaacc ttgggaaaatacactatatcttaaactccatgaaagaaggtgaggctgcaaacagctaatgcacattggcaacagcccctgatgcctat gccttattcatccctcagaaaaggattcaagtagaggcttgatttggaggttaaagttttgctatgctgtattttacattacttattgttttagct gtcctcatgaatgtcttttcactacccatttgcttatcctgcatctctcagccttgactccactcagttctcttgcttagagataccacctttccc ctgaagtgttccttccatgttttacggcgagatggtttctcctcgcctggccactcagccttagttgtctctgttgtcttatagaggtctacttg aagaaggaaaaacagggggcatggtttgactgtcctgtgagcccttcttccctgcctcccccactcacagtgacccggaatccctcga catggcagtctagcactagtgcggccgcagatctgcttcctcgctcactgactcgctgcgctcggtcgttcggctgcggcgagcggtat cagctcactcaaaggcggtaatacggttatccacagaatcaggggataacgcaggaaagaacatgtgagcaaaaggccagcaaaa ggccaggaaccgtaaaaaggccgcgttgctggcgtttttccataggctccgcccccctgacgagcatcacaaaaatcgacgctcaag tcagaggtggcgaaacccgacaggactataaagataccaggcgtttccccctggaagctccctcgtgcgctctcctgttccgaccctg ccgcttaccggatacctgtccgcctttctcccttcgggaagcgtggcgctttctcatagctcacgctgtaggtatctcagttcggtgtaggt cgttcgctccaagctgggctgtgtgcacgaaccccccgttcagcccgaccgctgcgccttatccggtaactatcgtcttgagtccaacc cggtaagacacgacttatcgccactggcagcagccactggtaacaggattagcagagcgaggtatgtaggcggtgctacagagttctt gaagtggtggcctaactacggctacactagaagaacagtatttggtatctgcgctctgctgaagccagttaccttcggaaaaagagttg gtagctcttgatccggcaaacaaaccaccgctggtagcggtggtttttttgtttgcaagcagcagattacgcgcagaaaaaaaggatctc aagaagatcctttgatcttttctacggggtctgacgctcagtggaacgaaaactcacgttaagggattttggtcatgagattatcaaaaag gatcttcacctagatccttttaaattaaaaatgaagttttaaatcaatctaaagtatatatgagtaaacttggtctgacagttagaaaaactcat cgagcatcaaatgaaactgcaatttattcatatcaggattatcaataccatatttttgaaaaagccgtttctgtaatgaaggagaaaactcac cgaggcagttccataggatggcaagatcctggtatcggtctgcgattccgactcgtccaacatcaatacaacctattaatttcccctcgtc aaaaataaggttatcaagtgagaaatcaccatgagtgacgactgaatccggtgagaatggcaaaagtttatgcatttctttccagacttgt tcaacaggccagccattacgctcgtcatcaaaatcactcgcatcaaccaaaccgttattcattcgtgattgcgcctgagcgagacgaaat acgcgatcgctgttaaaaggacaattacaaacaggaatcgaatgcaaccggcgcaggaacactgccagcgcatcaacaatattttcac ctgaatcaggatattcttctaatacctggaatgctgtttttccggggatcgcagtggtgagtaaccatgcatcatcaggagtacggataaa atgcttgatggtcggaagaggcataaattccgtcagccagtttagtctgaccatctcatctgtaacatcattggcaacgctacctttgccat gtttcagaaacaactctggcgcatcgggcttcccatacaagcgatagattgtcgcacctgattgcccgacattatcgcgagcccatttat acccatataaatcagcatccatgttggaatttaatcgcggcctcgacgtttcccgttgaatatggctcataacaccccttgtattactgtttat gtaagcagacagttttattgttcatgatgatatatttttatcttgtgcaatgtaacatcagagattttgagacacgggccagagctgcacaca tttccccgaaaagtgccacctgacgt (SEQ ID NO: 2). In some embodiments, the second vector comprises the nucleic acid sequence of SEQ ID NO: 2. In some embodiments, the second vector consists of the nucleic acid sequence of SEQ ID NO: 2. In some embodiments, the second vector is depicted in the vector map shown in FIG. 2B and is referred to herein as the “MD2.G” vector.

In some embodiments, the third vector comprises a polynucleotide comprising GAG and POL. In some embodiments, the third vector also comprises a REV protein binding site. In some embodiments, the Gag gene encodes major structural proteins of viral particles, such as nucleocapsid protein, membrane protein and capsid protein. In some embodiments, the pol gene encodes viral replication-associated enzymes, such as protease, reverse transcriptase and integrase. In some embodiments, the third vector comprises a nucleic acid sequence that is at least 90%, at least 91%, at least 92%, at least 93%, at least 94%, at least 95%, at least 96%, at least 97%, at least 98%, or at least 99% identical to the following nucleic acid sequence: ggatcccctgagggggcccccatgggctagaggatccggcctcggcctctgcataaataaaaaaaattagtcagccatgagcttggc ccattgcatacgttgtatccatatcataatatgtacatttatattggctcatgtccaacattaccgccatgttgacattgattattgactagttatt aatagtaatcaattacggggtcattagttcatagcccatatatggagttccgcgttacataacttacggtaaatggcccgcctggctgacc gcccaacgacccccgcccattgacgtcaataatgacgtatgttcccatagtaacgccaatagggactttccattgacgtcaatgggtgg agtatttacggtaaactgcccacttggcagtacatcaagtgtatcatatgccaagtacgccccctattgacgtcaatgacggtaaatggc ccgcctggcattatgcccagtacatgaccttatgggactttcctacttggcagtacatctacgtattagtcatcgctattaccatggtgatgc ggttttggcagtacatcaatgggcgtggatagcggtttgactcacggggatttccaagtctccaccccattgacgtcaatgggagtttgtt ttggcaccaaaatcaacgggactttccaaaatgtcgtaacaactccgccccattgacgcaaatgggcggtaggcgtgtacggtggga ggtctatataagcagagctcgtttagtgaaccgtcagatcgcctggagacgccatccacgctgttttgacctccatagaagacaccggg accgatccagcctcccctcgaagcttacatgtggtaccgagctcggatcctgagaacttcagggtgagtctatgggacccttgatgtttt ctttccccttcttttctatggttaagttcatgtcataggaaggggagaagtaacagggtacacatattgaccaaatcagggtaattttgcattt gtaattttaaaaaatgetttettettttaatatacttttttgtttatettatttetaatactttccctaatetetttetttcagggcaataatgatacaatg tatcatgcctctttgcaccattctaaagaataacagtgataatttctgggttaaggcaatagcaatatttctgcatataaatatttctgcatata aattgtaactgatgtaagaggtttcatattgctaatagcagctacaatccagctaccattctgcttttattttatggttgggataaggctggatt attctgagtccaagctaggcccttttgctaatcatgttcatacctcttatcttcctcccacagctcctgggcaacgtgctggtctgtgtgctg gcccatcactttggcaaagcacgtgagatctgaattcgagatctgccgccgccatgggtgcgagagcgtcagtattaagcgggggag aattagatcgatgggaaaaaattcggttaaggccagggggaaagaaaaaatataaattaaaacatatagtatgggcaagcagggagct agaacgattcgcagttaatcctggcctgttagaaacatcagaaggctgtagacaaatactgggacagctacaaccatcccttcagacag gatcagaagaacttagatcattatataatacagtagcaaccctctattgtgtgcatcaaaggatagagataaaagacaccaaggaagctt tagacaagatagaggaagagcaaaacaaaagtaagaaaaaagcacagcaagcagcagctgacacaggacacagcaatcaggtca gccaaaattaccctatagtgcagaacatccaggggcaaatggtacatcaggccatatcacctagaactttaaatgcatgggtaaaagta gtagaagagaaggctttcagcccagaagtgatacccatgttttcagcattatcagaaggagccaccccacaagatttaaacaccatgct aaacacagtggggggacatcaagcagccatgcaaatgttaaaagagaccatcaatgaggaagctgcagaatgggatagagtgcatc cagtgcatgagggcctattgcaccaggccagatgagagaaccaaggggaagtgacatagcaggaactactagtacccttcaggaac aaataggatggatgacacataatccacctatcccagtaggagaaatctataaaagatggataatcctgggattaaataaaatagtaagaa tgtatagccctaccagcattctggacataagacaaggaccaaaggaaccctttagagactatgtagaccgattctataaaactctaagag ccgagcaagcttcacaagaggtaaaaaattggatgacagaaaccttgttggtccaaaatgcgaacccagattgtaagactattttaaaa gcattgggaccaggagcgacactagaagaaatgatgacagcatgtcagggagtggggggacccggccataaagcaagagttttgg ctgaagcaatgagccaagtaacaaatccagctaccataatgatacagaaaggcaattttaggaaccaaagaaagactgttaagtgtttc aattgtggcaaagaagggcacatagccaaaaattgcagggcccctaggaaaaagggctgttggaaatgtggaaaggaaggacacc aaatgaaagattgtactgagagacaggctaattttttagggaagatctggccttcccacaagggaaggccagggaattttcttcagagc agaccagagccaacagccccaccagaagagagcttcaggtttggggaagagacaacaactccctctcagaagcaggagccgatag acaaggaactgtatcctttagcttccctcagatcactctttggcagcgacccctcgtcacaataaagataggggggcaattaaaggaag ctctattagatacaggagcagatgatacagtattagaagaaatgaatttgccaggaagatggaaaccaaaaatgatagggggaattgg aggttttatcaaagtaagacagtatgatcagatactcatagaaatctgcggacataaagctataggtacagtattagtaggacctacacct gtcaacataattggaagaaatctgttgactcagattggctgcactttaaattttcccattagtcctattgagactgtaccagtaaaattaaag ccaggaatggatggcccaaaagttaaacaatggccattgacagaagaaaaaataaaagcattagtagaaatttgtacagaaatggaaa aggaaggaaaaatttcaaaaattgggcctgaaaatccatacaatactccagtatttgccataaagaaaaaagacagtactaaatggaga aaattagtagatttcagagaacttaataagagaactcaagatttctgggaagttcaattaggaataccacatcctgcagggttaaaacaga aaaaatcagtaacagtactggatgtgggcgatgcatatttttcagttcccttagataaagacttcaggaagtatactgcatttaccataccta gtataaacaatgagacaccagggattagatatcagtacaatgtgcttccacagggatggaaaggatcaccagcaatattccagtgtagc atgacaaaaatcttagagccttttagaaaacaaaatccagacatagtcatctatcaatacatggatgatttgtatgtaggatctgacttaga aatagggcagcatagaacaaaaatagaggaactgagacaacatctgttgaggtggggatttaccacaccagacaaaaaacatcagaa agaacctccattcctttggatgggttatgaactccatcctgataaatggacagtacagcctatagtgctgccagaaaaggacagctgga ctgtcaatgacatacagaaattagtgggaaaattgaattgggcaagtcagatttatgcagggattaaagtaaggcaattatgtaaacttctt aggggaaccaaagcactaacagaagtagtaccactaacagaagaagcagagctagaactggcagaaaacagggagattctaaaag aaccggtacatggagtgtattatgacccatcaaaagacttaatagcagaaatacagaagcaggggcaaggccaatggacatatcaaat ttatcaagagccatttaaaaatctgaaaacaggaaagtatgcaagaatgaagggtgcccacactaatgatgtgaaacaattaacagagg cagtacaaaaaatagccacagaaagcatagtaatatggggaaagactcctaaatttaaattacccatacaaaaggaaacatgggaagc atggtggacagagtattggcaagccacctggattcctgagtgggagtttgtcaatacccctcccttagtgaagttatggtaccagttaga gaaagaacccataataggagcagaaactttctatgtagatggggcagccaatagggaaactaaattaggaaaagcaggatatgtaact gacagaggaagacaaaaagttgtccccctaacggacacaacaaatcagaagactgagttacaagcaattcatctagctttgcaggatt cgggattagaagtaaacatagtgacagactcacaatatgcattgggaatcattcaagcacaaccagataagagtgaatcagagttagtc agtcaaataatagagcagttaataaaaaaggaaaaagtctacctggcatgggtaccagcacacaaaggaattggaggaaatgaacaa gtagataaattggtcagtgctggaatcaggaaagtactatttttagatggaatagataaggcccaagaagaacatgagaaatatcacagt aattggagagcaatggctagtgattttaacctaccacctgtagtagcaaaagaaatagtagccagctgtgataaatgtcagctaaaagg ggaagccatgcatggacaagtagactgtagcccaggaatatggcagctagattgtacacatttagaaggaaaagttatcttggtagcag ttcatgtagccagtggatatatagaagcagaagtaattccagcagagacagggcaagaaacagcatacttcctcttaaaattagcagga agatggccagtaaaaacagtacatacagacaatggcagcaatttcaccagtactacagttaaggccgcctgttggtgggcggggatca agcaggaatttggcattccctacaatccccaaagtcaaggagtaatagaatctatgaataaagaattaaagaaaattataggacaggtaa gagatcaggctgaacatcttaagacagcagtacaaatggcagtattcatccacaattttaaaagaaaaggggggattggggggtacag tgcaggggaaagaatagtagacataatagcaacagacatacaaactaaagaattacaaaaacaaattacaaaaattcaaaattttcggg tttattacagggacagcagagatccagtttggaaaggaccagcaaagctcctctggaaaggtgaaggggcagtagtaatacaagata atagtgacataaaagtagtgccaagaagaaaagcaaagatcatcagggattatggaaaacagatggcaggtgatgattgtgtggcaa gtagacaggatgaggattaacacatggaattccggagcggccgcaggagctttgttccttgggttcttgggagcagcaggaagcacta tgggcgcagcgtcaatgacgctgacggtacaggccagacaattattgtctggtatagtgcagcagcagaacaatttgctgagggctatt gaggcgcaacagcatctgttgcaactcacagtctggggcatcaagcagctccaggcaagaatcctggctgtggaaagatacctaaag gatcaacagctcctggggatttggggttgctctggaaaactcatttgcaccactgctgtgccttggaatgctagttggagtaataaatctct ggaacagatttggaatcacacgacctggatggagtgggacagagaaattaacaattacacaagcttccgcggaattcaccccaccagt gcaggctgcctatcagaaagtggtggctggtgtggctaatgccctggcccacaagtatcactaagctcgctttcttgctgtccaatttcta ttaaaggttcctttgttccctaagtccaactactaaactgggggatattatgaagggccttgagcatctggattctgcctaataaaaaacatt tattttcattgcaatgatgtatttaaattatttctgaatattttactaaaaagggaatgtgggaggtcagtgcatttaaaacataaagaaatgaa gagctagttcaaaccttgggaaaatacactatatcttaaactccatgaaagaaggtgaggctgcaaacagctaatgcacattggcaaca gcccctgatgcctatgccttattcatccctcagaaaaggattcaagtagaggcttgatttggaggttaaagttttgctatgctgtattttacat tacttattgttttagctgtcctcatgaatgtcttttcactacccatttgcttatcctgcatctctcagccttgactccactcagttctcttgcttaga gataccacctttcccctgaagtgttccttccatgttttacggcgagatggtttctcctcgcctggccactcagccttagttgtctctgttgtctt atagaggtctacttgaagaaggaaaaacagggggcatggtttgactgtcctgtgagcccttcttccctgcctcccccactcacagtgac ccggaatccctcgacatggcagtctagcactagtgcggccgcagatctgcttcctcgctcactgactcgctgcgctcggtcgttcggct gcggcgagcggtatcagctcactcaaaggcggtaatacggttatccacagaatcaggggataacgcaggaaagaacatgtgagcaa aaggccagcaaaaggccaggaaccgtaaaaaggccgcgttgctggcgtttttccataggctccgcccccctgacgagcatcacaaa aatcgacgctcaagtcagaggtggcgaaacccgacaggactataaagataccaggcgtttccccctggaagctccctcgtgcgctct cctgttccgaccctgccgcttaccggatacctgtccgcctttctcccttcgggaagcgtggcgctttctcatagctcacgctgtaggtatct cagttcggtgtaggtcgttcgctccaagctgggctgtgtgcacgaaccccccgttcagcccgaccgctgcgccttatccggtaactatc gtcttgagtccaacccggtaagacacgacttatcgccactggcagcagccactggtaacaggattagcagagcgaggtatgtaggcg gtgctacagagttcttgaagtggtggcctaactacggctacactagaagaacagtatttggtatctgcgctctgctgaagccagttacctt cggaaaaagagttggtagctcttgatccggcaaacaaaccaccgctggtagcggtggtttttttgtttgcaagcagcagattacgcgca gaaaaaaaggatctcaagaagatcctttgatcttttctacggggtctgacgctcagtggaacgaaaactcacgttaagggattttggtcat gagattatcaaaaaggatcttcacctagatccttttaaattaaaaatgaagttttaaatcaatctaaagtatatatgagtaaacttggtctgac agttagaaaaactcatcgagcatcaaatgaaactgcaatttattcatatcaggattatcaataccatatttttgaaaaagccgtttctgtaatg aaggagaaaactcaccgaggcagttccataggatggcaagatcctggtatcggtctgcgattccgactcgtccaacatcaatacaacc tattaatttcccctcgtcaaaaataaggttatcaagtgagaaatcaccatgagtgacgactgaatccggtgagaatggcaaaagtttatgc atttctttccagacttgttcaacaggccagccattacgctcgtcatcaaaatcactcgcatcaaccaaaccgttattcattcgtgattgcgcc tgagcgagacgaaatacgcgatcgctgttaaaaggacaattacaaacaggaatcgaatgcaaccggcgcaggaacactgccagcg catcaacaatattttcacctgaatcaggatattcttctaatacctggaatgctgtttttccggggatcgcagtggtgagtaaccatgcatcat caggagtacggataaaatgcttgatggtcggaagaggcataaattccgtcagccagtttagtctgaccatctcatctgtaacatcattgg caacgctacctttgccatgtttcagaaacaactctggcgcatcgggcttcccatacaagcgatagattgtcgcacctgattgcccgacatt atcgcgagcccatttatacccatataaatcagcatccatgttggaatttaatcgcggcctcgacgtttcccgttgaatatggctcataacac cccttgtattactgtttatgtaagcagacagttttattgttcatgatgatatatttttatcttgtgcaatgtaacatcagagattttgagacacgg gccagagctgcacacatttccccgaaaagtgccacctgacgt (SEQ ID NO: 3). In some embodiments, the third vector comprises the nucleic acid sequence of SEQ ID NO: 3. In some embodiments, the third vector consists of the nucleic acid sequence of SEQ ID NO: 3. In some embodiments, the third vector is depicted in the vector map shown in FIG. 2C and is referred to herein as the “VIDLg” vector.

In some embodiments, the fourth vector comprises a polynucleotide encoding REV. In some embodiments, the Rev gene controls expression levels of the Gag and Pol genes and guiding the replication process of the single-stranded DNA and can regulate splicing/RNA transport. In some embodiments, the fourth vector comprises a nucleic acid sequence that is at least 90%, at least 91%, at least 92%, at least 93%, at least 94%, at least 95%, at least 96%, at least 97%, at least 98%, or at least 99% identical to the following nucleic acid sequence: gtaatacaaggggtgttatgagccatattcaacgggaaacgtcgaggccgcgattaaattccaacatggatgctgatttatatgggtata aatgggctcgcgataatgtcgggcaatcaggtgcgacaatctatcgcttgtatgggaagcccgatgcgccagagttgtttctgaaacat ggcaaaggtagcgttgccaatgatgttacagatgagatggtcagactaaactggctgacggaatttatgcctcttccgaccatcaagcat tttatccgtactcctgatgatgcatggttactcaccactgcgatccccggaaaaacagcattccaggtattagaagaatatcctgattcag gtgaaaatattgttgatgcgctggcagtgttcctgcgccggttgcattcgattcctgtttgtaattgtccttttaacagcgatcgcgtatttcgt ctcgctcaggcgcaatcacgaatgaataacggtttggttgatgcgagtgattttgatgacgagcgtaatggctggcctgttgaacaagtc tggaaagaaatgcataaacttttgccattctcaccggattcagtcgtcactcatggtgatttctcacttgataaccttatttttgacgagggg aaattaataggttgtattgatgttggacgagtcggaatcgcagaccgataccaggatcttgccatcctatggaactgcctcggtgagtttt ctccttcattacagaaacggctttttcaaaaatatggtattgataatcctgatatgaataaattgcagtttcatttgatgctcgatgagtttttcta actgtcagaccaagtttactcatatatactttagattgatttaaaacttcatttttaatttaaaaggatctaggtgaagatcctttttgataatctc atgaccaaaatcccttaacgtgagttttcgttccactgagcgtcagaccccgtagaaaagatcaaaggatcttcttgagatcctttttttctg cgcgtaatctgctgcttgcaaacaaaaaaaccaccgctaccagcggtggtttgtttgccggatcaagagctaccaactctttttccgaag gtaactggcttcagcagagcgcagataccaaatactgttcttctagtgtagccgtagttaggccaccacttcaagaactctgtagcaccg cctacatacctcgctctgctaatcctgttaccagtggctgctgccagtggcgataagtcgtgtcttaccgggttggactcaagacgatagt taccggataaggcgcagcggtcgggctgaacggggggttcgtgcacacagcccagcttggagcgaacgacctacaccgaactgag atacctacagcgtgagctatgagaaagcgccacgcttcccgaagggagaaaggcggacaggtatccggtaagcggcagggtcgga acaggagagcgcacgagggagcttccagggggaaacgcctggtatctttatagtcctgtcgggtttcgccacctctgacttgagcgtc gatttttgtgatgctcgtcaggggggcggagcctatggaaaaacgccagcaacgcggcctttttacggttcctggccttttgctggccttt tgctcacatgttctttcctgcgttatcccctgattctgtggataaccgtattaccgcctttgagtgagctgataccgctcgccgcagccgaa cgaccgagcgcagcgagtcagtgagcgaggaagcggaagagcgcccaatacgcaaaccgcctctccccgcgcgttggccgattc attaatgcagctggcacgacaggtttcccgactggaaagcgggcagtgagcgcaacgcaattaatgtgagttagctcactcattaggc accccaggctttacactttatgcttccggctcgtatgttgtgtggaattgtgagcggataacaatttcacacaggaaacagctatgacatg attacgaattcgatgtacgggccagatatacgcgtatctgaggggactagggtgtgtttaggcgaaaagcggggcttcggttgtacgc ggttaggagtcccctcaggatatagtagtttcgcttttgcatagggagggggaaatgtagtcttatgcaatactcttgtagtcttgcaacat ggtaacgatgagttagcaacatgccttacaaggagagaaaaagcaccgtgcatgccgattggtggaagtaaggtggtacgatcgtgc cttattaggaaggcaacagacgggtctgacatggattggacgaaccactgaattccgcattgcagagatattgtatttaagtgcctagct cgatacaataaacgccatttgaccattcaccacattggtgtgcacctccaagctcgagctcgtttagtgaaccgtcagatcgcctggaga cgccatccacgctgttttgacctccatagaagacaccgggaccgatccagcctcccctcgaagctagtcgattaggcatctcctatggc aggaagaagcggagacagcgacgaagacctcctcaaggcagtcagactcatcaagtttctctatcaaagcaacccacctcccaatcc cgaggggacccgacaggcccgaaggaatagaagaagaaggtggagagagagacagagacagatccattcgattagtgaacggat ccttagcacttatctgggacgatctgcggagcctgtgcctcttcagctaccaccgcttgagagacttactcttgattgtaacgaggattgt ggaacttctgggacgcagggggtgggaagccctcaaatattggtggaatctcctacaatattggagtcaggagctaaagaatagtgct gttagcttgctcaatgccacagctatagcagtagctgaggggacagatagggttatagaagtagtacaagaagcttggcactggccgt cgttttacaacgtcgtgatctgagcctgggagatctctggctaactagggaacccactgcttaagcctcaataaagcttgccttgagtgct tcaagtagtgtgtgcccgtctgttgtgtgactctggtaactagagatcaggaaaaccctggcgttacccaacttaatcgccttgcagcac atccccctttcgccagctggcgtaatagcgaagaggcccgcaccgatcgcccttcccaacagttgcgcagcctgaatggcgaatggc gcctgatgcggtattttctccttacgcatctgtgcggtatttcacaccgcatacgtcaaagcaaccatagtacgcgccctgtagcggcgc attaagcgcggcgggtgtggtggttacgcgcagcgtgaccgctacacttgccagcgccctagcgcccgctcctttcgctttcttcccttc ctttctcgccacgttcgccggctttccccgtcaagctctaaatcgggggctccctttagggttccgatttagtgctttacggcacctcgacc ccaaaaaacttgatttgggtgatggttcacgtagtgggccatcgccctgatagacggtttttcgccctttgacgttggagtccacgttcttt aatagtggactcttgttccaaactggaacaacactcaaccctatctcgggctattcttttgatttataagggattttgccgatttcggcctatt ggttaaaaaatgagctgatttaacaaaaatttaacgcgaattttaacaaaatattaacgtttacaattttatggtgcactctcagtacaatctg ctctgatgccgcatagttaagccagccccgacacccgccaacacccgctgacgcgccctgacgggcttgtctgctcccggcatccgc ttacagacaagctgtgaccgtctccgggagctgcatgtgtcagaggttttcaccgtcatcaccgaaacgcgcgagacgaaagggcct cgtgatacgcctatttttataggttaatgtcatgataataatggtttcttagacgtcaggtggcacttttcggggaaatgtg (SEQ ID NO: 4). In some embodiments, the fourth vector comprises the nucleic acid sequence of SEQ ID NO: 4. In some embodiments, the fourth vector consists of the nucleic acid sequence of SEQ ID NO: 4. In some embodiments, the fourth vector is depicted in the vector map shown in FIG. 2D and is referred to herein as the “RSV-REV” vector.

In some embodiments, the transfection media is selected from any suitable transfection media or medium, for example, T-cell growth media (TCGM) or CTS™ LV-MAX™ Production Medium (Thermo Fischer).

In some embodiments, the first vector (“GOI”), the second vector (“MD2.G”), the third vector (“MDLg”), and the fourth vector (“RSV-REV”) are included in the transfection composition in a ratio. In some embodiments, the ratio between the first vector, second vector, third vector, and fourth vector are present at a ratio of A:B:C:D, wherein A represents the value of the first vector, B represents the value of the second vector, C represents the value of the third vector, and D represents the value of the fourth vector. In some embodiments, A represents a value from 2 to about 16, B represents a value from about 1 to about 5, C represents a value from about 1 to about 5, and D represents a value from about 1 to about 5.

In some embodiments, the first vector is present in the ratio at a value range of about 2 to about 16. In some embodiments, the first vector is present in the ratio at a value of about 2. In some embodiments, the first vector is present in the ratio at a value of about 3. In some embodiments, the first vector is present in the ratio at a value of about 4. In some embodiments, the first vector is present in the ratio at a value of about 5. In some embodiments, the first vector is present in the ratio at a value of about 6. In some embodiments, the first vector is present in the ratio at a value of about 7. In some embodiments, the first vector is present in the ratio at a value of about 8. In some embodiments, the first vector is present in the ratio at a value of about 9. In some embodiments, the first vector is present in the ratio at a value of about 10. In some embodiments, the first vector is present in the ratio at a value of about 11. In some embodiments, the first vector is present in the ratio at a value of about 12. In some embodiments, the first vector is present in the ratio at a value of about 13. In some embodiments, the first vector is present in the ratio at a value of about 14. In some embodiments, the first vector is present in the ratio at a value of about 15. In some embodiments, the first vector is present in the ratio at a value of about 16.

In some embodiments, the second vector is present in the ratio at a value range of about 1 to about 5. In some embodiments, the second vector is present in the ratio at a value of about 2. In some embodiments, the second vector is present in the ratio at a value of about 3. In some embodiments, the second vector is present in the ratio at a value of about 4. In some embodiments, the second vector is present in the ratio at a value of about 5.

In some embodiments, the third vector is present in the ratio at a value range of about 1 to about 5. In some embodiments, the third vector is present in the ratio at a value of about 2. In some embodiments, the third vector is present in the ratio at a value of about 3. In some embodiments, the third vector is present in the ratio at a value of about 4. In some embodiments, the third vector is present in the ratio at a value of about 5.

In some embodiments, the fourth vector is present in the ratio at a value range of about 1 to about 5. In some embodiments, the fourth vector is present in the ratio at a value of about 2. In some embodiments, the fourth vector is present in the ratio at a value of about 3. In some embodiments, the fourth vector is present in the ratio at a value of about 4. In some embodiments, the fourth vector is present in the ratio at a value of about 5.

In some embodiments, he first vector, second vector, third vector, and fourth vector are present at a ratio of: 2-16:1-5:1-5:1-5, such as 2-11:1-3:1:1-5, 2-10:1-3:1:1-5, 2-9:1-3:1:1-5, 2-8:1-3:1:1-5, 2-7:1-3:1:1-5, 2-6:1-3:1:1-5, 2-5:1-3:1:1-5, 2-4:1-3:1:1-5, 2-3:1-3:1:1-5, 2-7:1-3:1:1-5, 3-12:1-3:1:1-5, 3-11:1-3:1:1-5, 3-10:1-3:1:1-5, 3-9:1-3:1:1-5, 3-8:1-3:1:1-5, 3-7:1-3:1:1-5, 3-6:1-3:1:1-5, 3-5:1-3:1:1-5, 3-4:1-3:1:1-5, 4-12:1-3:1:1-5, 4-11:1-3:1:1-5, 4-10:1-3:1:1-5, 4-9:1-3:1:1-5, 4-8:1-3:1:1-5, 4-7:1-3:1:1-5, 4-6:1-3:1:1-5, 4-5:1-3:1:1-5, 5-12:1-3:1:1-5, 5-11:1-3:1:1-5, 5-10:1-3:1:1-5, 5-9:1-3:1:1-5, 5-8:1-3:1:1-5, 5-7:1-3:1:1-5, 5-6:1-3:1:1-5, 6-12:1-3:1:1-5, 7-12:1-3:1:1-5, 7-11:1-3:1:1-5, 7-10:1-3:1:1-5, 7-9:1-3:1:1-5, 7-8:1-3:1:1-5, 8-12:1-3:1:1-5, 8-11:1-3:1:1-5, 8-10:1-3:1:1-5, 8-9:1-3:1:1-5, 9-12:1-3:1:1-5, 9-11:1-3:1:1-5, 9-10:1-3:1:1-5, 10-12:1-3:1:1-5, 10-11:1-3:1:1-5, 11-12:1-3:1:1-5. In some embodiments, the first vector, second vector, third vector, and fourth vector are present at a ratio of: 2-12:1-3:1:1-5, such as: 2-12:1-2:1:1-5, 2-12:2-3:1:1-5. In some embodiments, the first vector, second vector, third vector, and fourth vector are present at a ratio of: 2-12:1-3:1:1-5, such as: 2-12:1-3:1:1-4, 2-12:1-3:1:1-3, 2-12:1-3:1:1-2, 2-12:1-3:1:2-5, 2-12:1-3:1:2-4, 2-12:1-3:1:2-3, 2-12:1-3:1:3-5, 2-12:1-3:1:3-4, 2-12:1-3:1:4-5.

In some embodiments, the first vector, the second vector, the third vector, and the fourth vector are present at a ratio of 6:4:5:5, 6:5:4:5, 6:5:5:4, 8:2:5:5, 8:3:4:5, 8:3:5:4, 8:4:4:4, 8:4:5:3, 8:5:2:5, 8:5:3:4, 8:5:4:3, 8:5:5:2, 9:5:1:5, 10:1:4:5, 10:1:5:4, 10:2:3:5, 10:2:4:4, 10:2:5:3, 10:3:2:5, 10:3:5:2, 10:4:1:5, 10:4:2:4, 10:4:4:2, 10:4:5:1, 10:5:1:4, 10:5:2:3, 10:5:3:2, 10:5:4:1, 11:3:1:5, 12:1:2:5, 12:1:3:4, 12:1:4:3, 12:1:5:2, 12:2:1:5, 12:2:2:4, 12:2:5:1, 12:3:1:4, 12:3:4:1, 12:4:1:3, 12:4:2:2, 12:4:3:1, 12:5:1:2, 12:5:2:1, 13:1:1:5, 14:1:1:4, 14:1:2:3, 14:1:3:2, 14:1:4:1, 14:2:1:3, 14:2:3:1, 14:3:1:2, 14:3:2:1, 14:4:1:1, 16:1:1:2, 16:1:2:1, or 16:2:1:1. In some embodiments, the first vector, the second vector, the third vector, and the fourth vector are present at a ratio of 6:4:5:5. In some embodiments, the first vector, the second vector, the third vector, and the fourth vector are present at a ratio of 6:5:4:5. In some embodiments, the first vector, the second vector, the third vector, and the fourth vector are present at a ratio of 6:5:5:4. In some embodiments, the first vector, the second vector, the third vector, and the fourth vector are present at a ratio of 8:2:5:5. In some embodiments, the first vector, the second vector, the third vector, and the fourth vector are present at a ratio of 8:3:4:5. In some embodiments, the first vector, the second vector, the third vector, and the fourth vector are present at a ratio of 8:3:5:4. In some embodiments, the first vector, the second vector, the third vector, and the fourth vector are present at a ratio of 8:4:4:4. In some embodiments, the first vector, the second vector, the third vector, and the fourth vector are present at a ratio of 8:4:5:3. In some embodiments, the first vector, the second vector, the third vector, and the fourth vector are present at a ratio of 8:5:2:5. In some embodiments, the first vector, the second vector, the third vector, and the fourth vector are present at a ratio of 8:5:3:4. In some embodiments, the first vector, the second vector, the third vector, and the fourth vector are present at a ratio of 8:5:4:3. In some embodiments, the first vector, the second vector, the third vector, and the fourth vector are present at a ratio of 8:5:5:2. In some embodiments, the first vector, the second vector, the third vector, and the fourth vector are present at a ratio of 9:5:1:5. In some embodiments, the first vector, the second vector, the third vector, and the fourth vector are present at a ratio of 10:1:4:5. In some embodiments, the first vector, the second vector, the third vector, and the fourth vector are present at a ratio of 10:1:5:4. In some embodiments, the first vector, the second vector, the third vector, and the fourth vector are present at a ratio of 10:2:3:5. In some embodiments, the first vector, the second vector, the third vector, and the fourth vector are present at a ratio of 10:2:4:4. In some embodiments, the first vector, the second vector, the third vector, and the fourth vector are present at a ratio of 10:2:5:3. In some embodiments, the first vector, the second vector, the third vector, and the fourth vector are present at a ratio of 10:3:2:5. In some embodiments, the first vector, the second vector, the third vector, and the fourth vector are present at a ratio of 10:3:5:2. In some embodiments, the first vector, the second vector, the third vector, and the fourth vector are present at a ratio of 10:4:1:5. In some embodiments, the first vector, the second vector, the third vector, and the fourth vector are present at a ratio of 10:4:2:4. In some embodiments, the first vector, the second vector, the third vector, and the fourth vector are present at a ratio of 10:4:4:2. In some embodiments, the first vector, the second vector, the third vector, and the fourth vector are present at a ratio of 10:4:5:1. In some embodiments, the first vector, the second vector, the third vector, and the fourth vector are present at a ratio of 10:5:1:4. In some embodiments, the first vector, the second vector, the third vector, and the fourth vector are present at a ratio of 10:5:2:3. In some embodiments, the first vector, the second vector, the third vector, and the fourth vector are present at a ratio of 10:5:3:2. In some embodiments, the first vector, the second vector, the third vector, and the fourth vector are present at a ratio of 10:5:4:1. In some embodiments, the first vector, the second vector, the third vector, and the fourth vector are present at a ratio of 11:3:1:5. In some embodiments, the first vector, the second vector, the third vector, and the fourth vector are present at a ratio of 12:1:2:5. In some embodiments, the first vector, the second vector, the third vector, and the fourth vector are present at a ratio of 12:1:3:4. In some embodiments, the first vector, the second vector, the third vector, and the fourth vector are present at a ratio of 12:1:4:3. In some embodiments, the first vector, the second vector, the third vector, and the fourth vector are present at a ratio of 12:1:5:2. In some embodiments, the first vector, the second vector, the third vector, and the fourth vector are present at a ratio of 12:2:1:5. In some embodiments, the first vector, the second vector, the third vector, and the fourth vector are present at a ratio of 12:2:2:4. In some embodiments, the first vector, the second vector, the third vector, and the fourth vector are present at a ratio of 12:2:5:1. In some embodiments, the first vector, the second vector, the third vector, and the fourth vector are present at a ratio of 12:3:1:4. In some embodiments, the first vector, the second vector, the third vector, and the fourth vector are present at a ratio of 12:3:4:1. In some embodiments, the first vector, the second vector, the third vector, and the fourth vector are present at a ratio of 12:4:1:3. In some embodiments, the first vector, the second vector, the third vector, and the fourth vector are present at a ratio of 12:4:2:2. In some embodiments, the first vector, the second vector, the third vector, and the fourth vector are present at a ratio of 12:4:3:1. In some embodiments, the first vector, the second vector, the third vector, and the fourth vector are present at a ratio of 12:5:1:2. In some embodiments, the first vector, the second vector, the third vector, and the fourth vector are present at a ratio of 12:5:2:1. In some embodiments, the first vector, the second vector, the third vector, and the fourth vector are present at a ratio of 13:1:1:5. In some embodiments, the first vector, the second vector, the third vector, and the fourth vector are present at a ratio of 14:1:1:4. In some embodiments, the first vector, the second vector, the third vector, and the fourth vector are present at a ratio of 14:1:2:3. In some embodiments, the first vector, the second vector, the third vector, and the fourth vector are present at a ratio of 14:1:3:2. In some embodiments, the first vector, the second vector, the third vector, and the fourth vector are present at a ratio of 14:1:4:1. In some embodiments, the first vector, the second vector, the third vector, and the fourth vector are present at a ratio of 14:2:1:3. In some embodiments, the first vector, the second vector, the third vector, and the fourth vector are present at a ratio of 14:2:3:1. In some embodiments, the first vector, the second vector, the third vector, and the fourth vector are present at a ratio of 14:3:1:2. In some embodiments, the first vector, the second vector, the third vector, and the fourth vector are present at a ratio of 14:3:2:1. In some embodiments, the first vector, the second vector, the third vector, and the fourth vector are present at a ratio of 14:4:1:1. In some embodiments, the first vector, the second vector, the third vector, and the fourth vector are present at a ratio of 16:1:1:2. In some embodiments, the first vector, the second vector, the third vector, and the fourth vector are present at a ratio of 16:1:2:1. In some embodiments, the first vector, the second vector, the third vector, and the fourth vector are present at a ratio of 16:2:1:1.

In some embodiments, the first vector, second vector, third vector, and fourth vector are present in a ratio of 2:1:1:1, 11:3:1:5, or 12:2:1:5. In some embodiments, the first vector, second vector, third vector, and fourth vector is present in a ratio of 2:1:1:1. In some embodiments, the ratio between the first vector, second vector, third vector, and fourth vector is present in a ratio of 11:3:1:5. In some embodiments, the ratio between the first vector, second vector, third vector, and fourth vector is present in a ratio of 12:2:1:5.

### Methods

In some embodiments, preparing a lentivirus for the manufacture of a chimeric antigen receptor (CAR) T-cell (CAR-T) drug product is provided. In some embodiments, the method comprises transfecting host cells with a transfection composition, culturing the transfected host cells to proliferate, and harvesting the lentivirus. In some embodiments, the transfection composition is any transfection composition provided for herein. In some embodiments, the transfection composition comprises a first vector, a second vector, a third vector, and a fourth vector. In some embodiments, the vectors of the transfection composition are present in a ratio. In some embodiments the vector ratio is any vector ratio provided for herein.

In some embodiments, the time from transfection of the host cells to the harvesting of the lentivirus is less than or equal to about 48 hours. In some embodiments, the time from transfection of the host cells to the harvesting of the lentivirus is less than or equal to about 36 hours. In some embodiments, the time from transfection of the host cells to the harvesting of the lentivirus is less than or equal to about 24 hours. In some embodiments, the time from transfection of the host cells to the harvesting of the lentivirus is 22-26 hours, 23-25 hours, 23-34 hours, 23.5 to 24.5 hours, or 24-25 hours. In some embodiments, the time from transfection of the host cells to the harvesting of the lentivirus is about 24 hours. In some embodiments, the time from transfection of the host cells to the harvesting of the lentivirus is 24 hours.

In some embodiments, the vector ratio is 2:1:1:1 and the harvesting occurs about 24-hours after transfection. In some embodiments, the vector ratio is 11:3:1:5 and the harvesting occurs about 48-hours after transfection. In some embodiments, the vector ratio is 11:3:1:5 and the harvesting occurs about 24-hours after transfection. In some embodiments, the vector ratio is 12:2:1:5 and the harvesting occurs about 48-hours after transfection. In some embodiments, the vector ratio is 12:2:1:5 and the harvesting occurs about 24-hours after transfection.

In some embodiments, the culturing occurs in a bioreactor. In some embodiments the bioreactor is a 2 L, 10 L, or 50 L bioreactor. In some embodiments, the bioreactor is a shake flask.

In some embodiments, a method of manufacturing a CAR-T drug product is provided. In some embodiments, the CAR-T drug product is a BCMA CAR-T drug product. In some embodiments, the method manufacturing a CAR-T drug product comprises providing activated T cells from apheresis material from a subject, contacting the T cells with lentivirus prepared by any method disclosed herein or by the use of any transfection composition disclosed herein, culturing the transfected T cells to proliferate, and harvesting the CAR-T drug product.

In some embodiments, the CAR-T cell drug product produced during performance of the method described herein express one or more chimeric antigen receptors (CARs) on their surface. Generally, CARs comprise an extracellular domain from a first protein (e.g., an antigen-binding protein), a transmembrane domain, and an intracellular signaling domain, e.g., a primary signaling domain and optionally one or more costimulatory domains. In preferred embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

The extracellular domains of the CARs bind to an antigen of interest. In some embodiments, the extracellular domain of the CAR comprises a receptor, or a portion of a receptor, that binds to said antigen. In some embodiments, the extracellular domain comprises, or is, an antibody or an antigen-binding portion thereof. In some embodiments, the extracellular domain comprises, or is, a single chain Fv (scFv) domain. The single-chain Fv domain can comprise, for example, a VL linked to VH by a flexible linker, wherein said VL and VH are from an antibody that binds said antigen. In some embodiments, the single-chain Fv domain can comprise two VHH domains.

In some embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-β8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In some embodiments, the TAA or TSA recognized by the extracellular domain of a CAR is integrin αvβ3 (CD61), galactin, or Ral-B.

In some embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ES0-1, NY-SAR-35, OY-TES-1, SPANXBI, SPA17, SSX, SYCPI, or TPTE.

In some embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In some embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29†BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAA0205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, pi5(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68†KP1, CO-029, FGF-5, G250, Ga733 (EpCAM), HTgp-175, M344, MA-50, MG7-Ag, MOV18, NB†70K, NY-CO-1, RCAS1, SDCCAGi6, TA-90, TAAL6, TAG72, TLP, or TPS. Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In some embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In some embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In some embodiments, the intracellular domain of a CAR is or comprises an intracellular domain or motif of a protein that is expressed on or proximal to the surface of T cells and triggers activation and/or proliferation of said T cells. Such a domain or motif can transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is or comprises a CD3ζ intracellular signaling domain; a CD3ζ intracellular signaling domain may be referred to as a primary signaling domain. In other specific embodiments, the intracellular domain (primary signaling domain) is from a lymphocyte receptor chain, a TCR/CD3 complex protein, an Fc receptor subunit or an IL-2 receptor subunit.

In some embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence or domain, a co-stimulatory CD28 polypeptide sequence or domain, a co-stimulatory OX40 (CD134) polypeptide sequence or domain, a co-stimulatory 4-1BB (CD137) polypeptide sequence or domain, or a co-stimulatory inducible T-cell costimulatory (ICOS) polypeptide sequence or domain, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In some embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

In some embodiments, the CAR immunospecifically targets B-cell maturation antigen (BCMA). In some embodiments, the CAR-T drug product that immunospecifically targets BCMA is ciltacabtagene autoleucel. “Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (BCMA CAR). Cilta-cel is described in, for example, U.S. Pat. Nos. 10,934,363 and 11,535,677, both of which are incorporated by reference in their entirety

In some embodiments, a method of treating multiple myeloma is provided, the method comprising administrating a CAR-T drug product prepared by any method disclosed herein.

### ENUMERATED EMBODIMENTS

The following examples are illustrative, but not limiting, of the compounds, compositions and methods described herein. Other suitable modifications and adaptations known to those skilled in the art are within the scope of the following embodiments.

1. A method of preparing a lentivirus for the manufacture of a chimeric antigen receptor (CAR) T-cell (CAR-T) drug product, the method comprising:
transfecting host cells with a transfection composition, wherein the transfection composition comprises:
   a first vector comprising a polynucleotide encoding a CAR,
   a second vector, a third vector, and a fourth vector, and
   a transfection media;
   wherein the ratio of the first vector to the second vector to the third vector to
   the fourth vector is 2:1:1:1, 11:3:1:5, or 12:2:1:5,
culturing the transfected host cells to proliferate, and
harvesting the lentivirus, wherein the harvesting occurs about 24-hours after transfection.

2. A method of preparing a lentivirus for the manufacture of a chimeric antigen receptor (CAR) T-cell (CAR-T) drug product, the method comprising:
transfecting host cells with a transfection composition, wherein the transfection composition comprises:
   a first vector comprising a polynucleotide encoding a CAR,
   a second vector, a third vector, and a fourth vector, and
   a transfection media;
   wherein the ratio of the first vector to the second vector to the third vector to
   the fourth vectors is 11:3:1:5 or 12:2:1:5,
culturing the transfected host cells to proliferate, and harvesting the lentivirus, wherein the harvesting occurs less than or equal to about 48-hours after transfection.

3. The method of embodiment 1, wherein the ratio is 2:1:1:1.

4. The method of embodiment 1 or 2, wherein the ratio is 11:3:1:5.

5. The method of embodiment 1 or 2, wherein the ratio is 12:2:1:5.

6. The method of any one of embodiments 3 to 5, wherein the harvesting occurs about 24-hours after transfection.

7. The method of any one of embodiments 1 to 6, wherein the first vector, second vector, third vector, and fourth vector comprise lentiviral vectors.

8. The method of embodiment 1, wherein the ratio is 2:1:1:1 and the harvesting occurs about 24-hours after transfection.

9. The method of embodiment 1, wherein the ratio is 11:3:1:5 and the harvesting occurs about 24-hours after transfection.

10. The method of embodiment 1, wherein the ratio is 12:2:1:5 and the harvesting occurs about 24-hours after transfection.

11. The method of any one of embodiments 1 to 10, wherein the second vector comprises a polynucleotide encoding a lentiviral envelope protein.

12. The method of embodiment 11, wherein the lentiviral envelope protein is vesicular stomatitis virus G (VSVG).

13. The method of any one of embodiments 1 to 12, wherein the third vector comprises a polynucleotide encoding GAG and POL.

14. The method of any one of embodiments 1 to 13, wherein the fourth vector comprises a polynucleotide encoding REV.

15. The method of any one of embodiments 1 to 14, wherein the culturing occurs in a bioreactor.

16. The method of embodiment 15, wherein the bioreactor is a 2 L, 10 L or 50 L bioreactor.

17. The method of embodiments 1-16, wherein the host cells are HEK 293 cells, such as BEK 293F cells.

18. The method of any one of embodiments 1 to 17, wherein the CAR immunospecifically targets B-cell maturation antigen (BCMA).

19. A method of preparing a lentivirus for the manufacture of a BCMA CAR-T drug product, the method comprising:
transfecting host cells with a transfection composition, wherein the transfection composition comprises:
   a first vector comprising a polynucleotide encoding a CAR that immunospecifically targets BCMA,
   a second vector, a third vector, and a fourth vector, and
   a transfection media;
   wherein the ratio of the first vector to the second vector to the third vector to
   the fourth vector is 2:1:1:1, 11:3:1:5, or 12:2:1:5,
culturing the transfected host cells to proliferate, and
harvesting the lentivirus, wherein the harvesting occurs about 24-hours after transfection.

20. A method of preparing a lentivirus for the manufacture of a BCMA CAR-T drug product, the method comprising:
transfecting host cells with a transfection composition, wherein the transfection composition comprises:
   a first vector comprising a polynucleotide encoding a CAR that immunospecifically targets BCMA,
   a second vector, a third vector, and a fourth vector, and
   a transfection media;
   wherein the ratio of the first vector to the second vector to the third vector to
   the fourth vector is 11:3:1:5 or 12:2:1:5,
culturing the transfected host cells to proliferate, and
harvesting the lentivirus, wherein the harvesting occurs less than or equal to about 48-hours after transfection.

21. The method of embodiment 19, wherein the ratio is 2:1:1:1.

22. The method of embodiment 19 or 20, wherein the ratio is 11:3:1:5.

23. The method of embodiment 19 or 20, wherein the ratio is 12:2:1:5.

524. The method of any one of embodiments 20 to 23, wherein the harvesting occurs about 24-hours after transfection.

25. The method of any one of embodiments 19 to 24, wherein the first vector, second vector, third vector, and fourth vector comprise lentiviral vectors.

26. The method of embodiment 19, wherein the ratio is 2:1:1:1 and the harvesting occurs about 24-hours after transfection.

27. The method of embodiment 19, wherein the ratio is 11:3:1:5 and the harvesting occurs about 24-hours after transfection.

28. The method of embodiment 19, wherein the ratio is 12:2:1:5 and the harvesting occurs about 24-hours after transfection.

29. The method of any one of embodiments 19 to 28, wherein the second vector comprises a polynucleotide encoding a lentiviral envelope protein.

30. The method of embodiment 29, wherein the lentiviral envelope protein is vesicular stomatitis virus G (VSVG).

31. The method of any one of embodiments 19 to 30, wherein the third vector comprises a polynucleotide encoding GAG and POL.

32. The method of any one of embodiments 19 to 31, wherein the fourth vector comprises a polynucleotide encoding REV.

33. The method of any one of embodiments 19 to 32, wherein the culturing occurs in a bioreactor.

34. The method of embodiment 33, wherein the bioreactor is a 2 L, 10 L or 50 L bioreactor.

35. The method of any one of embodiments 19 to 34, wherein the host cells are HEK 293 cells, such as HEK 293F cells.

36. The method of any one of embodiments 19 to 35, wherein the first vector comprises SEQ ID NO: 1.

37. The method of any one of embodiments 19 to 36, wherein the second vector comprises SEQ ID NO: 2.

38. The method of any one of embodiments 19 to 37, wherein the third vector comprises SEQ ID NO: 3.

39. The method of any one of embodiments 19 to 38, wherein the fourth vector comprises SEQ ID NO: 4.

40. The method of any one of embodiment 19 to 39, wherein the CAR-T drug product is ciltacabtagene autolucel (cilta-cel).

41. A transfection composition, comprising:
a first vector comprising a polynucleotide encoding a CAR,
a second vector, a third vector, and a fourth vector, and
a transfection media;
wherein the ratio of the first vector to the second vector to the third vector to the fourth vector is 2:1:1:1, 11:3:1:5, or 12:2:1:5.

42. The transfection composition of embodiment 41, wherein the ratio is 2:1:1:1.

43. The transfection composition of embodiment 41, wherein the ratio is 11:3:1:5.

44. The transfection composition of embodiment 14, wherein the ratio is 12:2:1:5.

45. The transfection composition of any one of embodiments 41 to 44, wherein the second vector comprises a polynucleotide encoding a lentiviral envelope protein.

46. The transfection composition of embodiment 45, wherein the lentiviral envelope protein is vesicular stomatitis virus G (VSVG).

47. The transfection composition of any one of embodiments 41 to 46, wherein the third vector comprises a polynucleotide encoding GAG and POL.

48. The transfection composition of any one of embodiments 41 to 47, wherein the fourth vector comprises a polynucleotide encoding REV.

49. The transfection composition of any one of embodiments 41 to 48, wherein the CAR immunospecifically targets B-cell maturation antigen (BCMA).

50. The transfection composition of any one of embodiments 41 to 49, wherein the first vector comprises SEQ ID NO: 1.

51. The transfection composition of any one of embodiments 41 to 50, wherein the second vector comprises SEQ ID NO: 2.

52. The transfection composition of any one of embodiments 41 to 51, wherein the third vector comprises SEQ ID NO: 3.

53. The transfection composition of any one of embodiments 41 to 52, wherein the fourth vector comprises SEQ ID NO: 4.

54. A method of manufacturing a BCMA CAR-T drug product, the method comprising:
providing activated T cells from apheresis material from a subject having multiple myeloma,
contacting the T cells with lentivirus prepared by the method of any one of embodiments 1-40 or by the use of the transfection composition of any one of embodiments 41-53,
culturing the transfected T cells to proliferate, and
harvesting the CAR-T drug product.

55. The method of embodiment 54, wherein the BCMA CAR-T drug product is ciltacabtagene autolucel (cilta-cel).

56. A BCMA CAR-T drug product prepared by the method of claim 54 or 55.

57. A method of treating multiple myeloma, the method comprising administrating a BCMA CAR-T drug of claim 56 to a subject in need thereof.

58. A method for manufacturing a lentiviral vector for use in CAR-T cell therapy, comprising:
(a) Culturing host cells in a bioreactor at a specified temperature and medium;
(b) Transfecting the host cells with a transfection mix, wherein the transfection mix includes a transfer vector containing the gene of interest (GOI) and one or more helper vectors selected from MD2g, MDLg, and RSV-REV;
(c) Modifying the ratio of said vectors in the transfection mix, wherein the ratio of the vectors is adjusted to 11:3:1:5 (GOI:MD2g:MDLg) or 12:2:1:5;
(d) Harvesting the lentiviral vector after a culture period of 24 hours post-transfection.

59. The method of embodiment 58, wherein the host cells are HEK 293 cells cultured in a bioreactor with a working volume of 2 L, 10 L, or 50 L.

60. The method of embodiment 59, further comprising the step of adding a shear protectant and antifoaming agent to the bioreactor, wherein the shear protectant comprises a Kolliphor solution.

61. A method for optimizing lentiviral vector yield in CAR-T manufacturing, comprising:
(a) Transfecting cultured host cells with a mix of lentiviral vectors, including a GOI vector, MD2g, MDLg, and RSV-REV;
(b) Adjusting the ratio of the GOI vector and helper vectors to optimize infectious titer, wherein the ratio of GOI:MD2g:MDLg is adjusted to 11:3:1:5 or 12:2:1:5;
(c) Harvesting the cells 24 hours after transfection to reduce impurities and optimize titer recovery.

62. The method of embodiment 61, further comprising a purification process involving clarification, nuclease digestion, anion exchange purification, sterile filtration, and ultrafiltration/diafiltration (UF/DF).

63. A method for improving impurity profiles in lentiviral vector production, comprising:
(a) Culturing HEK 293 cells in a bioreactor;
(b) Transfecting the cells with a vector mix containing GOI, MD2g, MDLg, and RSV-REV vectors in a ratio of 11:3:1:5 or 12:2:1:5;
(c) Harvesting the cells 24 hours post-transfection to yield lentiviral vectors with reduced levels of p24, host cell proteins (HCP), and host cell DNA (HC-DNA) compared to a 48-hour post-transfection harvest.

64. The method of embodiment 63, wherein the harvested lentiviral vectors exhibit a reduction in p24 levels by 50% to 90% compared to a 48-hour harvest.

65. A lentiviral vector produced according to the method of embodiment 64, wherein the vector has improved transduction efficiency, reduced impurity levels, and enhanced quality attributes for use in CAR-T cell therapy.

66. A method for scaling up lentiviral vector production to a 50 L bioreactor, comprising:
(a) Performing upstream cell culture and transfection in a 50 L bioreactor;
(b) Transfecting the cells with a vector mix containing GOI, MD2g, MDLg, and RSV-REV in a ratio of 11:3:1:5;
(c) Harvesting the lentiviral vector 24 hours after transfection;
(d) Subjecting the harvested material to downstream purification, including clarification, nuclease digestion, anion exchange purification, and ultrafiltration/diafiltration.

67. The method of embodiment 66, wherein the infectious titers of lentiviral vectors produced at the 50 L scale are comparable to or higher than those produced at smaller scales.

### EXAMPLES

### Example 1: Modification of Vector Ratios for Manufacturing of CAR-T Drug Product

Manufacturing of a biological drug product, such as a CAR-T cell drug product, is a complex multi-day process. An essential step is providing the lentivirus to transduce enriched T cells to produce the CAR-T cell drug product. Manufacturing the lentivirus is itself a complex multi-day process that can be generally divided into upstream processing where the production is set up, and downstream processing where the lentivirus is harvested and prepared. A general overview of this entire lentiviral manufacturing process is depicted in the flow chat in FIG. 1. Without wishing to be bound by theory, upstream processing can be generally subdivided into two stages: 1) preculture and expansion, which can take up to 22 days, and 2) bioreactor production, which can take up to 4 days.

Stage 1 begins when a host cell material (here, HEK 293 cells are used) is thawed and cultured in a 250 mL shake flask for 4 days (passage 0). The cells are subsequently cultured in a 250 mL shake flask for 3 days (passage 1); a 500 mL shake flash for 4 days (passage 2); 2×1 L shake flasks for 3 days (passage 3); a 20 L wave bag for 4 days (passage 4); and a 50 L wave bag for 4 days (passage 5).

In stage 2, a 50 L bioreactor is used to culture the cells after passage 5 and produce lentivirus. One day before inoculation, the 50 L bioreactor is set up with media and temperature controls, and then on day 0, the 50 L bioreactor is inoculated with cells from passage 5. 10% Kolliphor solution is added immediately after inoculation as a shear protectant and antifoaming agent.. The cells are cultured until day 2, where the cell culture is transfected with a transfection mix that includes 4 different types of lentiviral vectors in a certain ratio. 4 hours after transfection, an anticlumping agent, a transfection enhancer, and supplements are added to the bioreactor. 48 hours after the transfection, the bioreactor is prepared for harvest and clarification (Stage 3, FIG. 1) and further downstream processing.

To determine if the harvest output can be increased, modifications to the lentiviral vector ratio were examined. In a lentiviral based vector product system, the natural viral genome is split into multiple helper vectors, typically four, which diminishes the risk of creating a replication-capable virus. The transfer vector contains the gene of interest (GOI), specifically the CAR coding sequence.. Also present are an envelope vector encoding VSVG (“MD2g”), a first packaging vector encoding GAG and POL (“MDLg”, and a second packaging vector encoding REV (“RSV-REV”). The vector map for the GOI vector is shown in FIG. 2A; the vector map for the MD2g vector is shown in FIG. 2B; the vector map for the MDLg vector is shown in FIG. 2C; and the vector map for the RSV-REV vector is show in FIG. 2D.

Normally, these vectors are present in the transfection mix in a 2:1:1:1 ratio (GOI:MD2g:MDLg:RSV-REV). Alternative vector ratios were designed and tested, using the following criteria: (1) total vector amount constant; (2) the amount of the transfer vector encoding the CAR just be greater than or equal to 5; (3) the amount of all other vectors must be greater than or equal to 1; and (4) the amount of transfer vector must be greater than the amount of any other vector.

Tables 1, 2, and 3 below list all the vector ratios tested. The study was executed into 3 blocks for easier testing. The various conditions were distributed in each block to cover the full range, so data is expected to show the same trend in each block. The analysis was conducted based on four quality attributes: 1) Infectious titer by SUPT1 (indication of infectious/working particles); 2) Infectious titer by AD TU Flow (indication of infectious/working particles); 3) p24 (indication of total infectious and non-infectious particles); and (4) P/I ratio (indicated of total particles per infectious/working particles).

TABLE 1
Tested Vector Ratios Group 1

| Block | RUN# | GOI | MD2g | MDLg | RSV-REV |
|---|---|---|---|---|---|
| 1 | 1 | 8 | 4 | 4 | 4 |
| 1 | 2 | 16 | 1 | 1 | 2 |
| 1 | 3 | 12 | 4 | 3 | 1 |
| 1 | 4 | 12 | 1 | 2 | 5 |
| 1 | 5 | 12 | 1 | 5 | 2 |
| 1 | 6 | 6 | 4 | 5 | 5 |
| 1 | 7 | 14 | 3 | 1 | 2 |
| 1 | 8 | 8 | 5 | 3 | 4 |
| 1 | 9 | 14 | 1 | 4 | 1 |
| 1 | 10 | 8 | 2 | 5 | 5 |
| 1 | 11 | 10 | 3 | 5 | 2 |
| 1 | 12 | 10 | 5 | 2 | 3 |
| 1 | 13 | 12 | 3 | 4 | 1 |
| 1 | 14 | 10 | 2 | 4 | 4 |
| 1 | 15 | 10 | 4 | 4 | 2 |
| 1 | 16 | 10 | 1 | 4 | 5 |
| 1 | 17 | 8 | 4 | 3 | 5 |
| 1 | 18 | 14 | 1 | 1 | 4 |

TABLE 2
Tested Vector Ratios Group 2

| Block | RUN# | GOI | MD2g | MDLg | RSV-REV |
|---|---|---|---|---|---|
| 2 | 21 | 8 | 4 | 4 | 4 |
| 2 | 22 | 10 | 5 | 3 | 2 |
| 2 | 23 | 8 | 4 | 5 | 3 |
| 2 | 24 | 8 | 5 | 4 | 3 |
| 2 | 25 | 12 | 5 | 1 | 2 |
| 2 | 26 | 16 | 1 | 2 | 1 |
| 2 | 27 | 10 | 1 | 5 | 4 |
| 2 | 28 | 12 | 4 | 1 | 3 |
| 2 | 29 | 10 | 4 | 1 | 5 |
| 2 | 30 | 10 | 4 | 5 | 1 |
| 2 | 31 | 14 | 2 | 1 | 3 |
| 2 | 32 | 12 | 2 | 1 | 5 |
| 2 | 33 | 6 | 5 | 4 | 5 |
| 2 | 34 | 14 | 3 | 2 | 1 |
| 2 | 35 | 12 | 1 | 3 | 4 |
| 2 | 36 | 14 | 1 | 2 | 3 |
| 2 | 37 | 12 | 2 | 5 | 1 |
| 2 | 38 | 10 | 2 | 5 | 3 |
| 2 | 39 | 8 | 3 | 4 | 5 |

TABLE 3
Tested Vector Ratios Group 3

| Block | RUN# | GOI | MD2g | MDLg | RSV-REV |
|---|---|---|---|---|---|
| 3 | 41 | 8 | 4 | 4 | 4 |
| 3 | 42 | 8 | 5 | 5 | 2 |
| 3 | 43 | 6 | 5 | 5 | 4 |
| 3 | 44 | 8 | 3 | 5 | 4 |
| 3 | 45 | 10 | 2 | 3 | 5 |
| 3 | 46 | 14 | 1 | 3 | 2 |
| 3 | 47 | 16 | 2 | 1 | 1 |
| 3 | 48 | 10 | 4 | 2 | 4 |
| 3 | 49 | 12 | 5 | 2 | 1 |
| 3 | 50 | 10 | 5 | 1 | 4 |
| 3 | 51 | 12 | 3 | 1 | 4 |
| 3 | 52 | 12 | 1 | 4 | 3 |
| 3 | 53 | 12 | 4 | 2 | 2 |
| 3 | 54 | 12 | 2 | 2 | 4 |
| 3 | 55 | 8 | 5 | 2 | 5 |
| 3 | 56 | 14 | 4 | 1 | 1 |
| 3 | 57 | 10 | 5 | 4 | 1 |
| 3 | 58 | 14 | 2 | 3 | 1 |
| 3 | 59 | 10 | 3 | 2 | 5 |
| 3 | 60 | 8 | 4 | 4 | 4 |

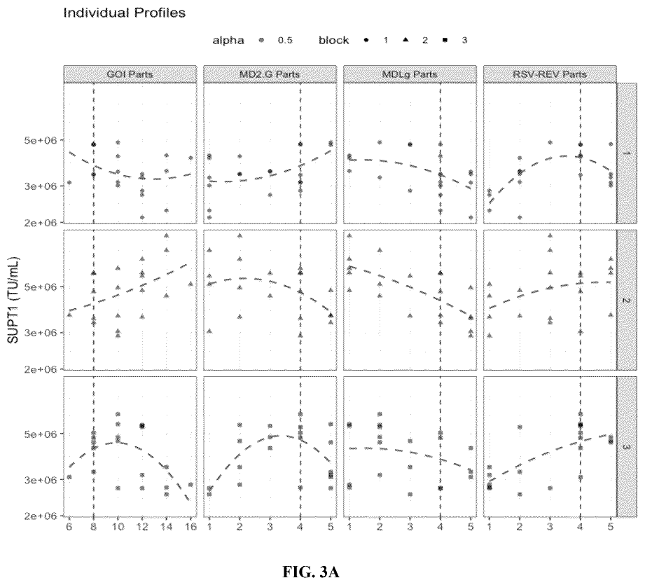

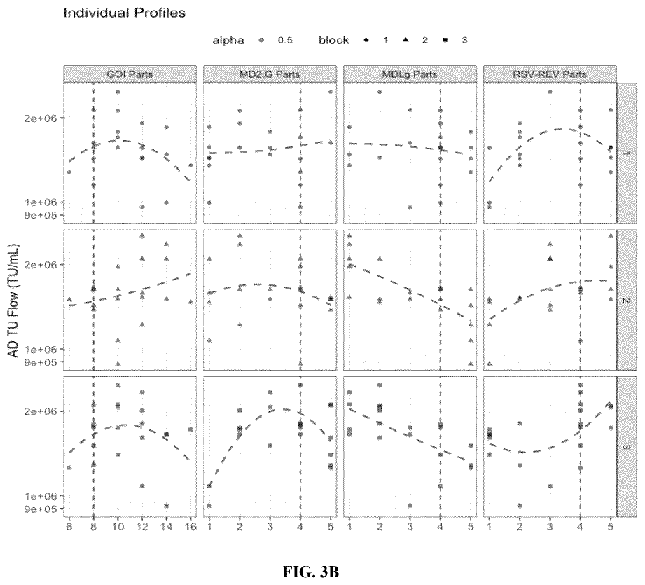

MDLg and RSV-REV vectors showed consistent trends across all blocks for both SUPT1 infectious titer (FIG. 3A) and AD TU Flow infectious titer (FIG. 3B). In contrast, conflicting trends were observed for the GOI and MD2G vectors.. Therefore, an additional experiment was designed to investigate the levels of the GOI and MD2G vectors while keeping the MDLg and RSV-REV vectors and their optimal levels.

Table 4 below lists the additional vector ratios tested. Except for the 8:4:4:4 control ratios in the first and final runs, the MDLg and RSV-REV levels were kept constant at 1 (MDLg) and either 4 or 5 (RSV-REV). GOI levels were varied from a low of 8 to a high of 14. MD2G levels were varied from a low of 1 to a high of 5.

TABLE 4
Additional Tested Vector Ratios

| Run | GOI Parts | MD2.G Parts | MDLg Parts | RSV-REV Parts |
|---|---|---|---|---|
| 1 | 8 | 4 | 4 | 4 |
| 2 | 9 | 5 | 1 | 5 |
| 3 | 14 | 1 | 1 | 4 |
| 4 | 11 | 3 | 1 | 5 |
| 5 | 14 | 1 | 1 | 4 |
| 6 | 12 | 2 | 1 | 5 |
| 7 | 10 | 4 | 1 | 5 |
| 8 | 13 | 1 | 1 | 5 |
| 9 | 11 | 3 | 1 | 5 |
| 10 | 9 | 5 | 1 | 5 |
| 11 | 13 | 1 | 1 | 5 |
| 12 | 9 | 5 | 1 | 5 |
| 13 | 10 | 4 | 1 | 5 |
| 14 | 12 | 2 | 1 | 5 |
| 15 | 13 | 1 | 1 | 5 |
| 16 | 14 | 1 | 1 | 4 |
| 17 | 12 | 2 | 1 | 5 |
| 18 | 11 | 3 | 1 | 5 |
| 19 | 10 | 4 | 1 | 5 |
| 20 | 8 | 4 | 4 | 4 |

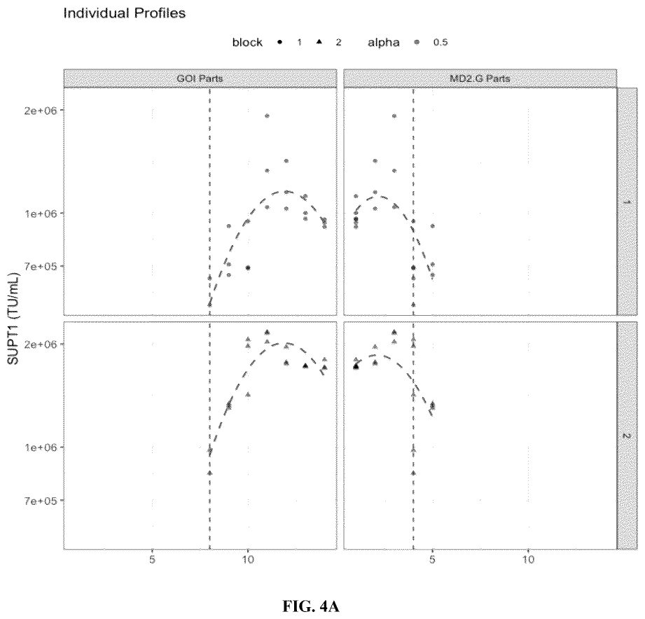

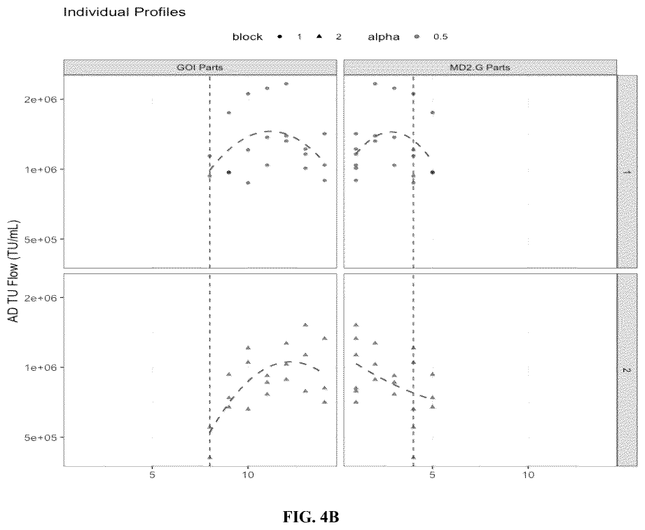

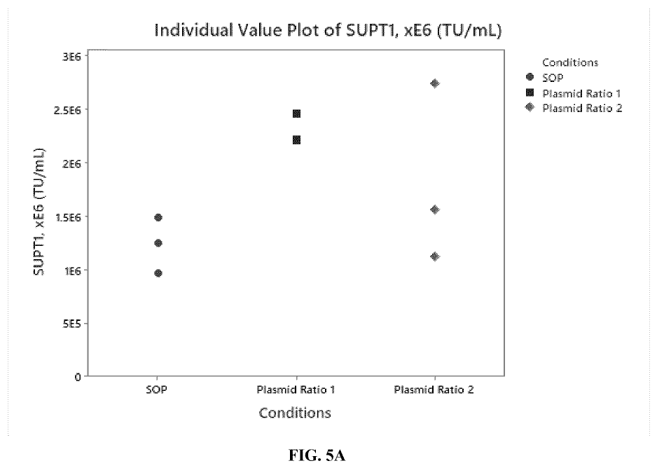

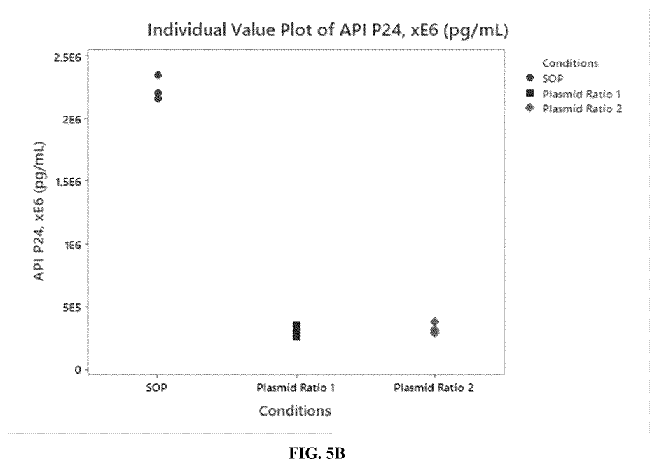

The analysis of these additional ratios was again conducted based on infectious titer by SUPT1 (indication of infectious/working particles, FIG. 4A) and infectious titer by AD TU Flow (indication of infectious/working particles, FIG. 4B). The entire block of ratios listed in Table 4 was repeated twice. The results showed that the vector ratios 11:3:1:5 and 12:2:1:5 appeared to be the strongest candidates. To confirm their improvement over the standard 2:1:1:1 vector ratio, confirmational testing runs were performed. Three runs each of the 2:1:1:1 control, 11:3:1:5, and 12:2:1:5 vector ratios were run, and both experimental ratios had higher titers (as shown by plots of SUPT1, xE6, FIG. 5A) and lower infectious particle ratios (as shown by plots of API P24, FIG. 5B) than the control ratio.

### Example 2: Modification of Harvest Time for Manufacturing of CAR-T Drug Product

As shown in FIG. 1, the cells are typically harvested and prepared for further downstream processing 48 hours after the cell culture is transfected. Without being bound by theory, it was hypothesized that the transfection process triggers a certain amount of cell death in the culture, which, as the dead cells rupture over time, can contribute to an increased in culture impurities. Additionally, the half-life of lentivirus at cell culture temperatures is relatively short, and after transfection, the rate at which infectious particles lose infectivity might outweigh the rates of infectious titer production by the cells. Thus, there might be an optimum time during which the titer remains similar or potentially higher to the titer recovered at 48 hours post transfection but may have lower levels of impurities.

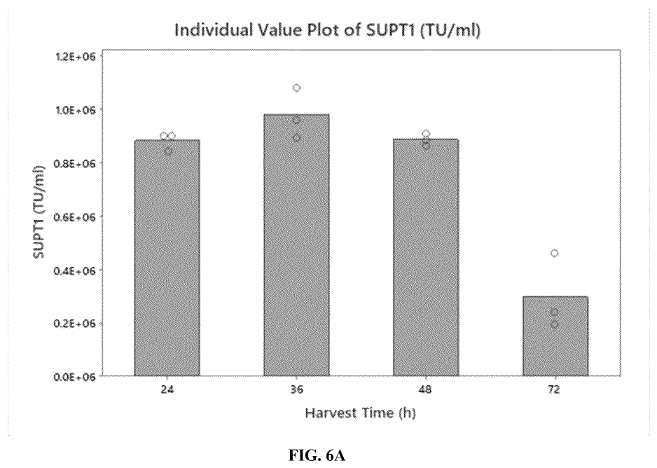

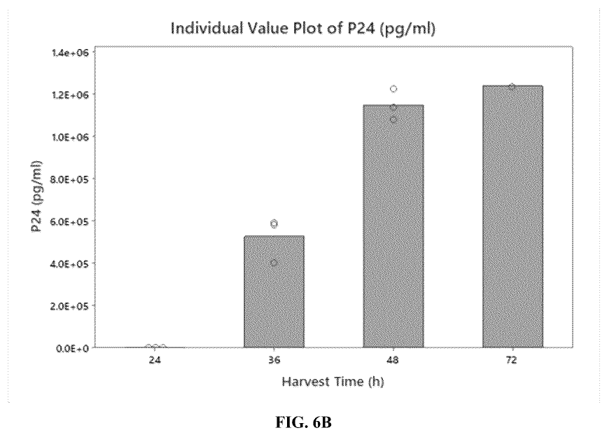

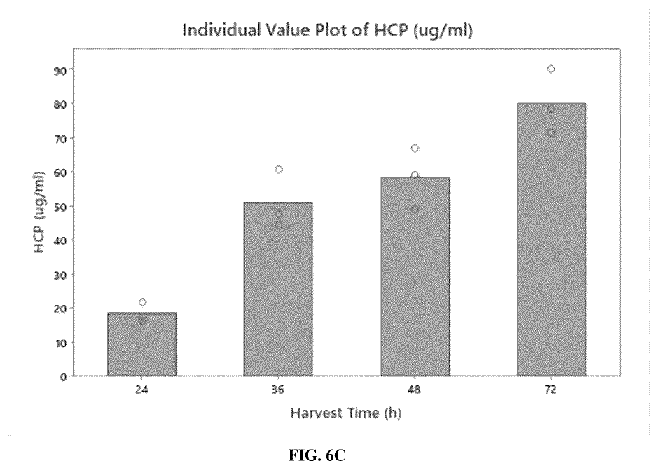

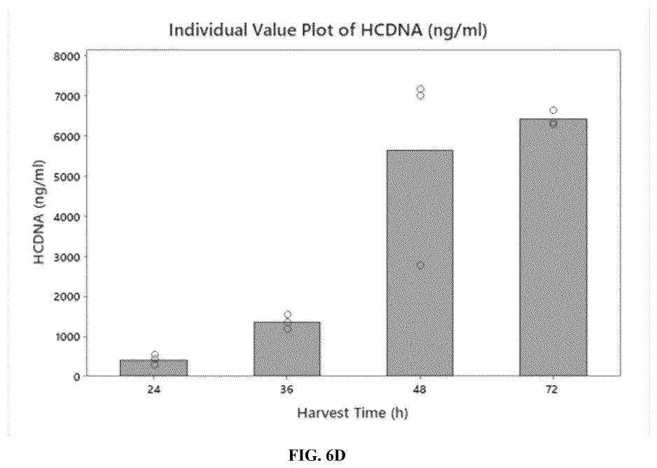

Thus, an experiment was run to examine harvest time conditions using a reduced scale model of the commercial 50 L process described in FIG. 1. The standard HEK293F cell line was used, as was the standard 2:1:1:1 lentiviral vector ratio (GOI:MD2g:MDLg:RSV-REV, see Example 1). Harvest times used were 24 hours, 36 hours, 48 hours (control), and 72 hours. The 24-hour and 36-hour harvest times resulted in similar infectious titers compared to the 48-hour control (FIG. 6A).. However, the 24-hour harvest time was significantly superior to all other harvest times in having lower levels of impurities: reduced physical particles at harvest (plot of P24, FIG. 6B), reduced host cell protein (HCP) at harvest (FIG. 6C), and reduced host cell DNA (HCDNA) at harvest (FIG. 6D).

### Example 3: Modification of Vector Ratios and Harvest Time for Manufacturing of CAR-T Drug Product

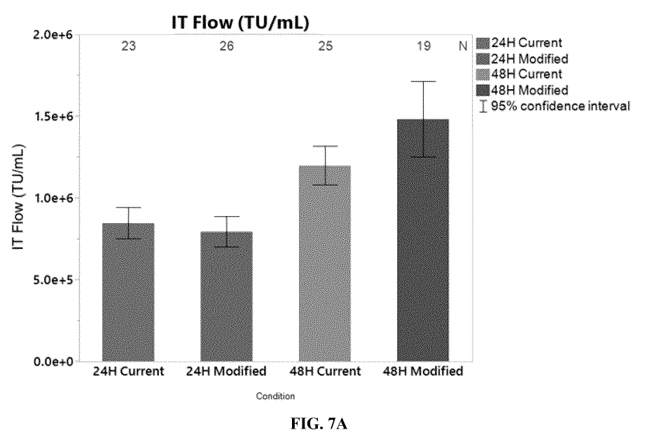

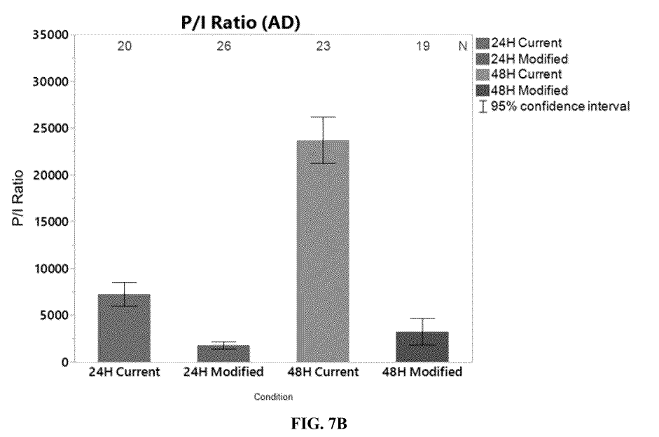

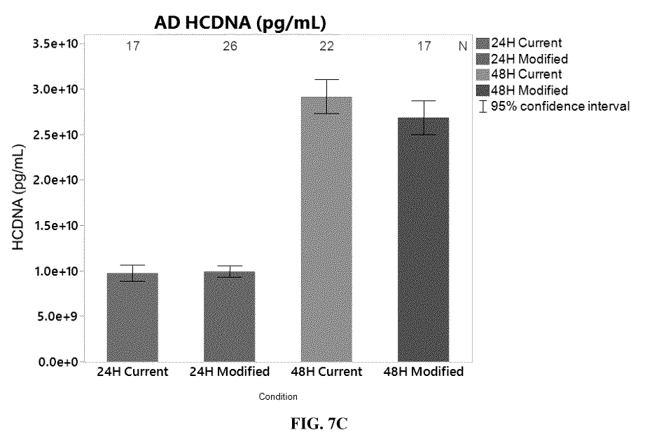

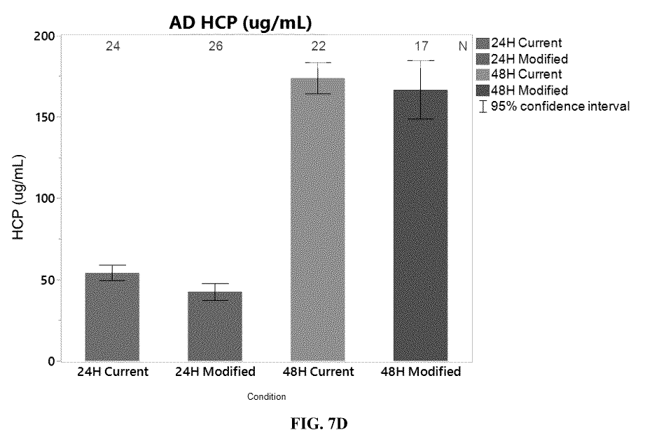

Taking into consideration the results of alternative lentiviral vector ratios (Example 1) and shorter harvest times (Example 2), harvest data was collected at various scales (e.g., 2 L and 10 L bioreactors), with a 24 hour harvest time with a 2:1:1:1 vector ratio (n=23), a 24 hour harvest time with a 11:3:1:5 vector ratio (n=26), a 48 hour harvest time with a 2:1:1:1 vector ratio (n=25), or a 48 hour harvest time with a 11:3:1:5 vector ratio (n=19). The results showed that the infectious titer trends slightly lower at harvest for both 24 hour harvest times (regardless of ratio), while the 48 hour, 11:3:1:5 vector ratio group showed the highest infectious titer (FIG. 7A). However, post-purification data (not shown) suggested there was no significant drop in titer with additional downstream steps. Nevertheless, testing showed that the 24-hour harvest time conditions had significantly lower levels of impurities, with the 24-hour, 11:3:1:5 vector ratio group having the lowest infectious particle ratio (infectious particles, FIG. 7B; HCDNA, FIG. 7C; HCP, FIG. 7D).

Based on these results, full end to end drug product runs using lentivirus manufactured by different processes were analyzed at harvest and post-downstream processing, and final drug product. The cell cultures were either 2 L or 10 L bioreactors, split into three groups: lentivirus manufactured with a 48-hour post transfection harvest time with a 2:1:1:1 vector ratio (control), lentivirus manufactured with a 24-hour post transfection harvest time with a 2:1:1:1 vector ratio, and lentivirus manufactured with a 24-hour post transfection harvest time with a 11:3:1:5 vector ratio. Additionally, this data was compared to a 50 L standard control. Once harvested, the downstream purification process for all conditions was clarified by depth filter Sartopure PP3 followed by Sartopore 2×LG filter train and underwent a nuclease digestion to remove vector and host cell DNA, AEX membrane-based purification and sterile filtration, and finally hollow fiber ultrafiltration/diafiltration (UF/DF). The drug product was then concentrated and formulated.

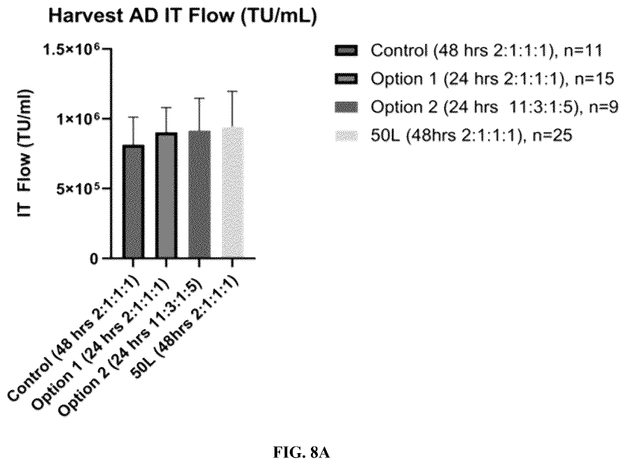

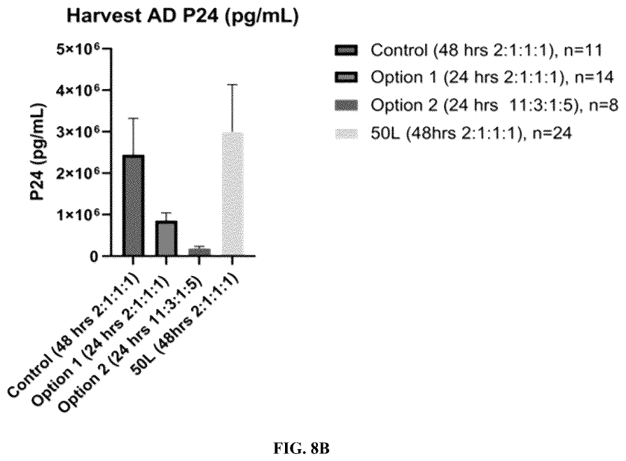

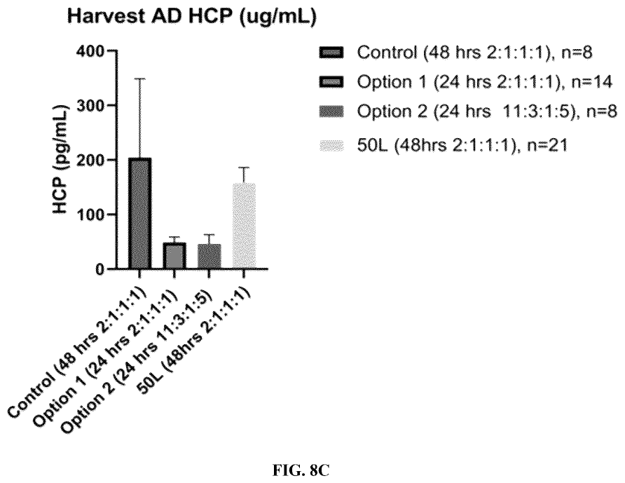

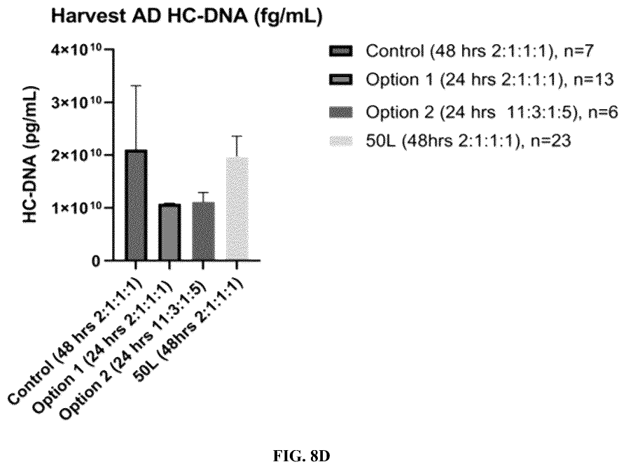

Upstream results at harvest are shown in FIGS. 8A-8D. The average infectious titer was relatively the same across all conditions (FIG. 8A), with the 24-hour conditions again showing markedly lower levels of P24, HCP, and HCDNA impurities than controls (FIGS. 8B, 8C, and 8D, respectively).

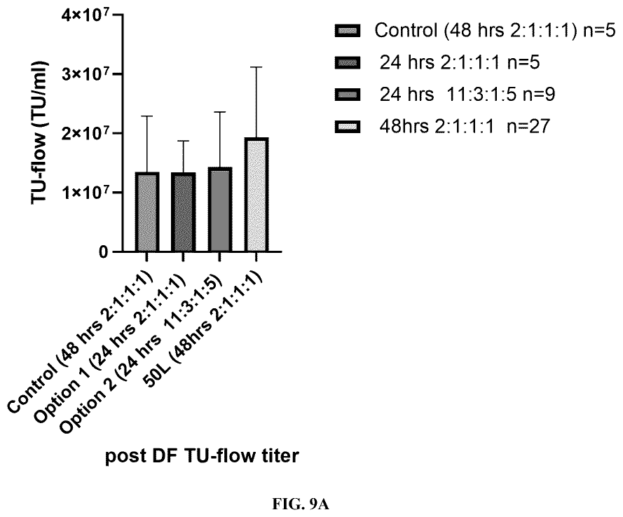

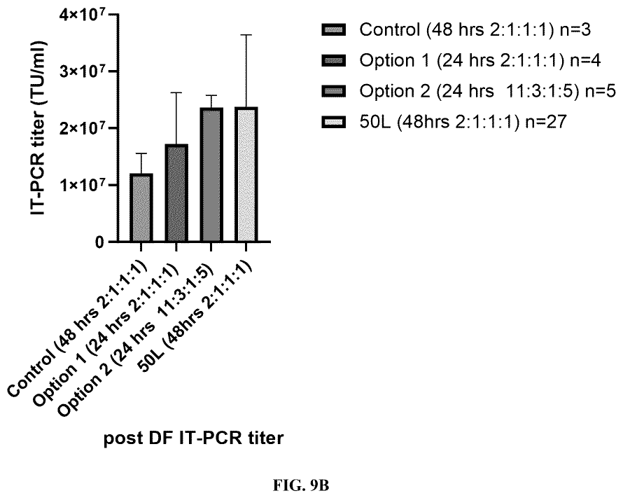

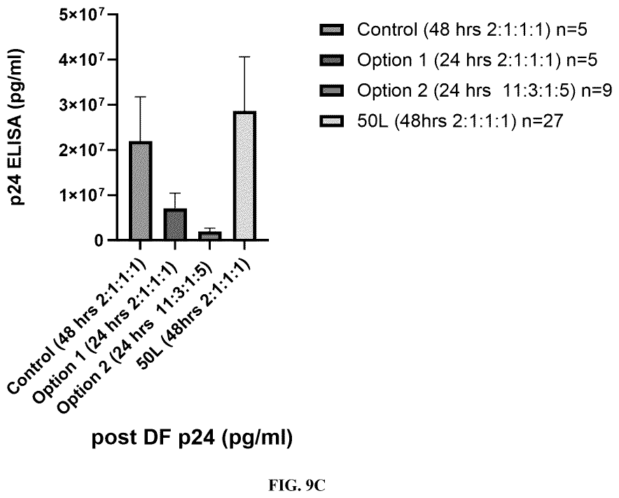

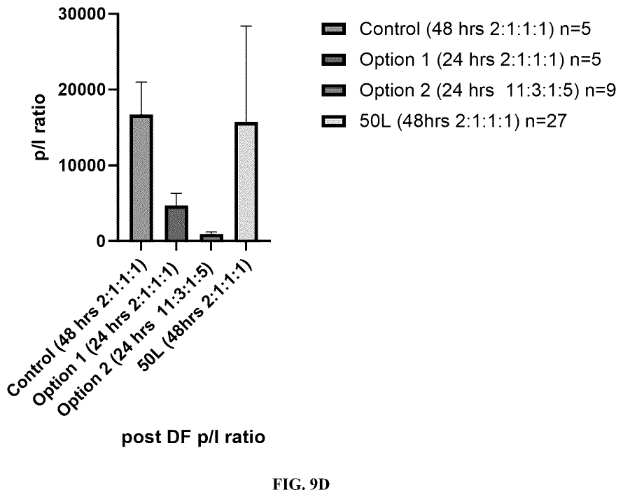

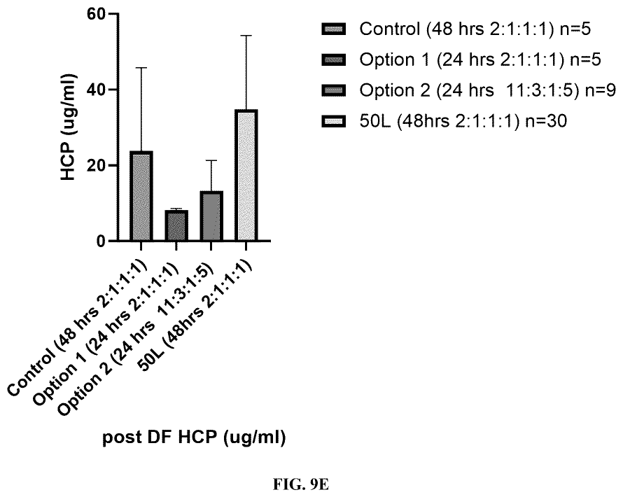

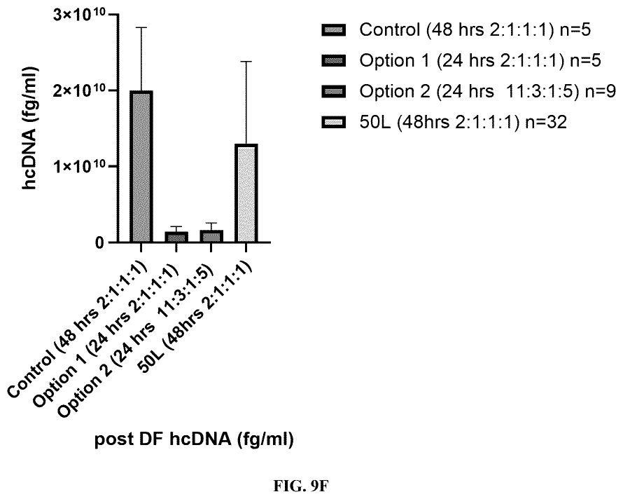

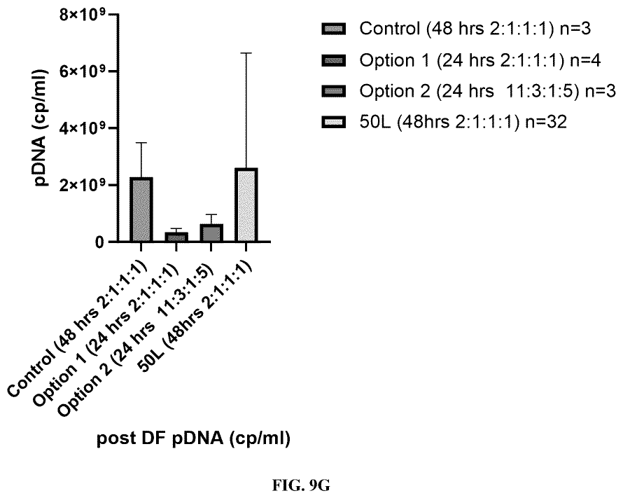

The downstream results post-downstream processing and formulations are shown in FIGS. 9A-9G. Both 24-hour conditions showed a similar infectious titer, just a bit lower than the full 50 L control (FIGS. 9A and 9B), and continued to show markedly lower levels of impurities:
the 24-hour, standard ratio condition showed about a 70% reduction of p24 levels compared to controls, and the 24-hour, 11:3:1:5 ratio condition showed about a 90% reduction of p24 levels (FIG. 9C)
the 24-hour, standard ratio condition showed about a 3.5-fold reduction of p/I ratio compared to controls, and the 24-hour, 11:3:1:5 ratio condition showed about a 17-fold reduction of p/I levels (FIG. 9D)
both 24-hour conditions have a HPC level more than 2-fold lower than controls (FIG. 9E)
both 24-hour conditions are more than one log lower in HCDNA levels compared to controls (FIG. 9F)
both 24-hours conditions have up to a 70% reduction in vector DNA levels compared to controls (FIG. 9G)
the 10 L control and the 50 L control conditions showed similar levels of impurities (FIGS. 9C-9G)

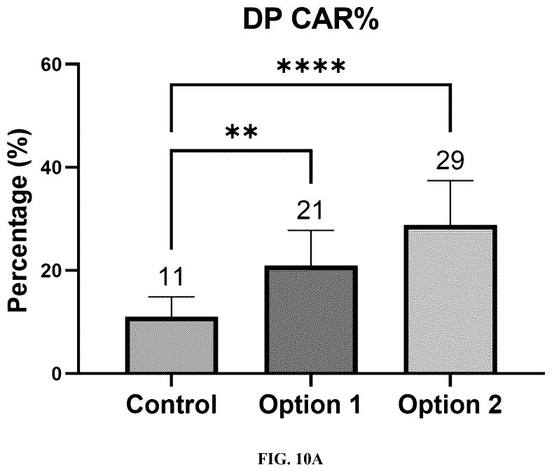

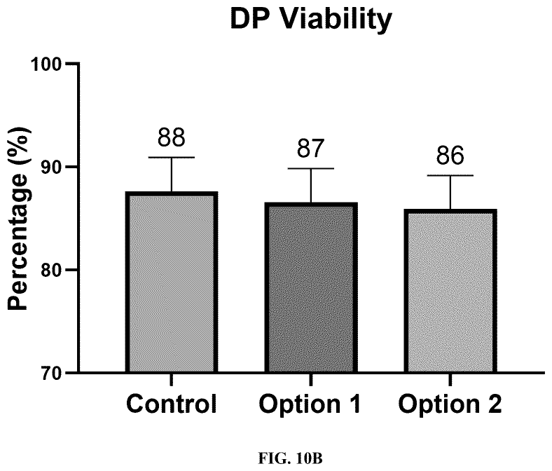

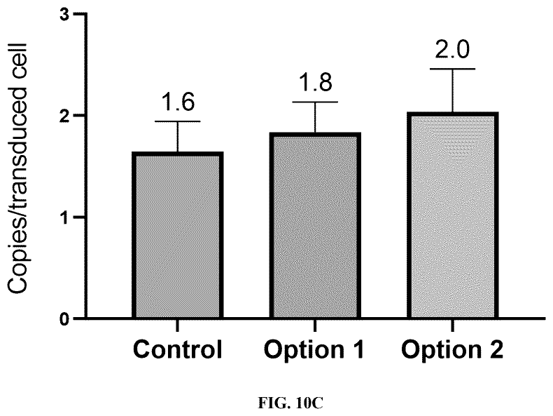

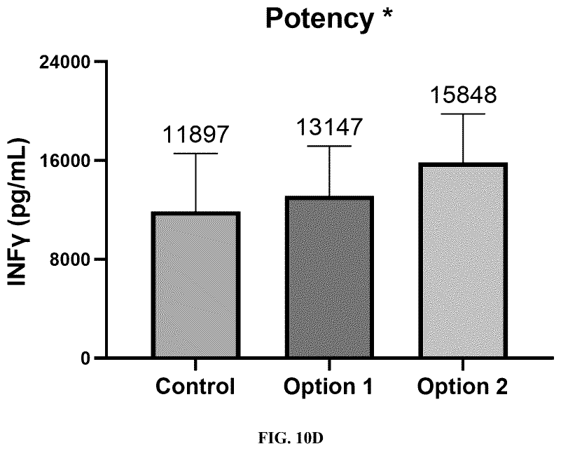

The final drug product results are shown in FIGS. 10A-10D. Compared to the standard control, a higher transduction efficiency was observed with either 24-hour condition. Both 24-hour conditions showed a higher CAR T cell percentage, with the 24-hour, 11:3:1:5 ratio condition having the highest levels (FIG. 10A). Drug product viability, vector copy number (VCN), and potency show no significant differences between the conditions (FIGS. 10B, 10C, and 10D respectively).

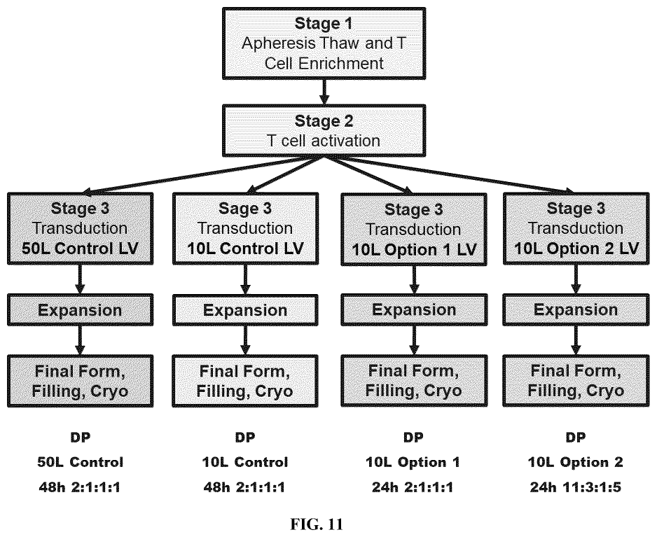

To fully confirm the above results, two full end-to-end drug product manufacturing runs were performed, with each run starting from one patient's apheresis sample. However, instead of just conducting only one transduction and expansion, each run contained four separate, but simultaneous transductions and expansions that differed only in how the lentivirus was manufactured: 1) a 50 L bioreactor, 48-hour post-transfection culture time, 2:1:1:1 vector ratio control group (50 L Control); 2) a 10 L bioreactor, 48-hour post-transfection culture time, 2:1:1:1 vector ratio control group (10 L Control); 3) a 10 L bioreactor, 24-hour post-transfection culture time, 2:1:1:1 vector ratio experimental group (10 L Option 1); and 4) a 10 L bioreactor, 24-hour post-transfection culture time, 11:3:1:5 vector ratio experimental group (10 L Option 2). A flow chart for one full end-to-end run with the four above groups is shown in FIG. 11.

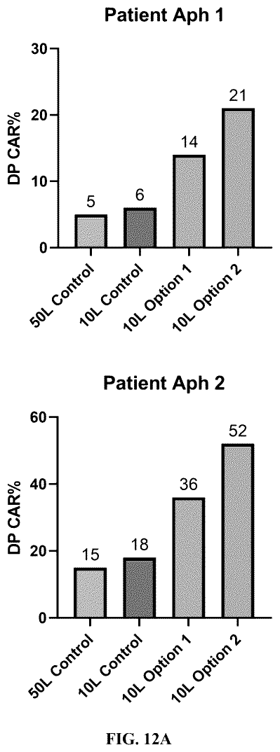

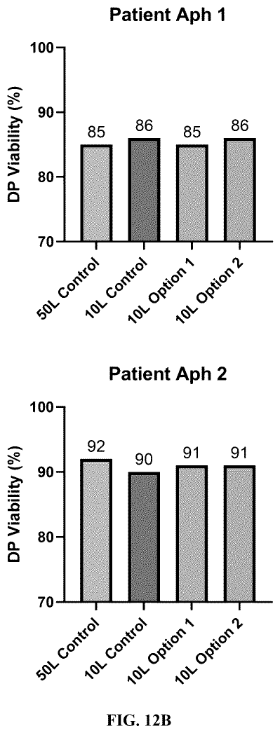

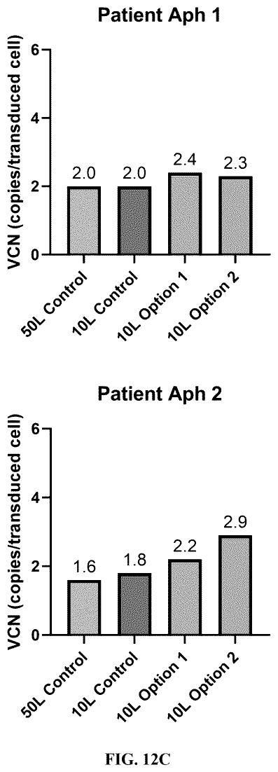

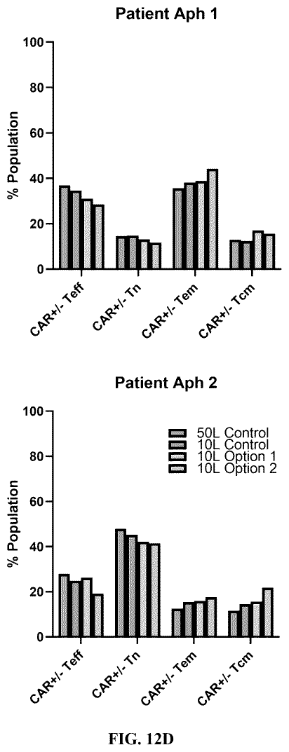

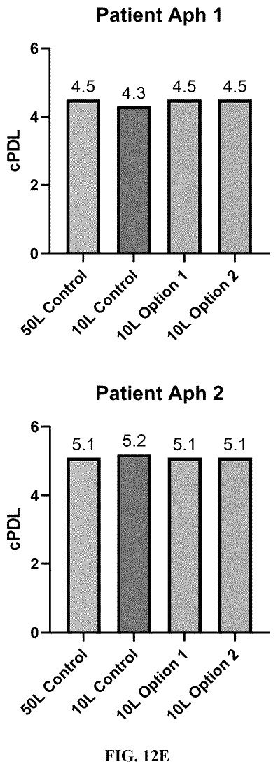

At the end of each run, the final drug product was analyzed, and the results are shown in FIGS. 12A-12E. The data from both runs are shown separately. Data shows higher transduction efficiency in both 24-hour groups, with the 11:3:1:5 vector ratio group performing best in both runs (FIG. 12A). There were no significant differences between the groups on drug product viability (FIG. 12B) and vector integration (VCN, FIG. 12C). Additionally, there was no obvious impact to various T cell immunophenotypes (FIG. 12D) or cell growth (FIG. 12E) across all conditions. Thus, either 24-hour group showed a higher drug product percentage without compromising anything compared to the both control groups, with the 11:3:1:5 vector ratio group showing the best performance.

### Example 4: 50 L Scale-Up in Lentiviral Manufacturing

The experiments conducted in Example 4 aimed to evaluate the performance and scalability of lentiviral vector (LVV) manufacturing processes at a 50 L bioreactor scale. In lentiviral manufacturing, scaling up from smaller bioreactor volumes is critical to meeting the production demands of CAR-T cell therapies. The manufacturing process, as previously described in Examples 1-3, involves multi-stage procedures that were initially tested and optimized at smaller scales, specifically 2 L and 10 L bioreactors. Examples 1-3 demonstrated that modifications to vector ratios and harvest times impact the quality and yield of LVVs, influencing both the infectious titer and impurity levels in the final product.

Building on these findings, the scale-up to a 50 L bioreactor aimed to verify whether the improvements observed at smaller scales could be maintained or enhanced in larger production runs. Here, the effects of different harvest times (24 hours vs. 48 hours post-transfection) and vector ratios (2:1:1:1 vs. 11:3:1:5) are assessed on key parameters such as infectious titer, impurity levels, and overall LVV product quality. The objective was to ensure that the process remained robust and efficient at the 50 L scale, thereby providing a reliable method for large-scale production of lentiviral vectors for CAR-T cell drug products.

Three distinct experimental conditions were evaluated in this study:
Control: A 48-hour post-transfection harvest time with a 2:1:1:1 vector ratio, tested at all three scales (2 L, 10 L, and 50 L);
Option 1: A 24-hour post-transfection harvest time while maintaining the same 2:1:1:1 vector ratio, tested at all three scales (2 L, 10 L, and 50 L);
Option 2: A 24-hour post-transfection harvest time with a modified 11:3:1:5 vector ratio, assessed only at the 2 L and 10 L scales.

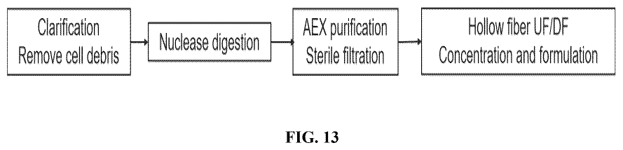

Following upstream bioreactor operations, the harvested material underwent a downstream purification process, as depicted in FIG. 13. The initial step involved clarification to remove cell debris, using a Sartopore PP3 depth filter followed by a Sartopore 2×LG filter train. This was followed by nuclease digestion to degrade residual plasmid and host cell DNA. The clarified and digested material was then subjected to anion exchange (AEX) membrane-based purification and sterile filtration to remove any remaining impurities. The final step involved ultrafiltration/diafiltration (UF/DF) using hollow fiber technology, concentrating the LVV and formulating it into the final drug substance.

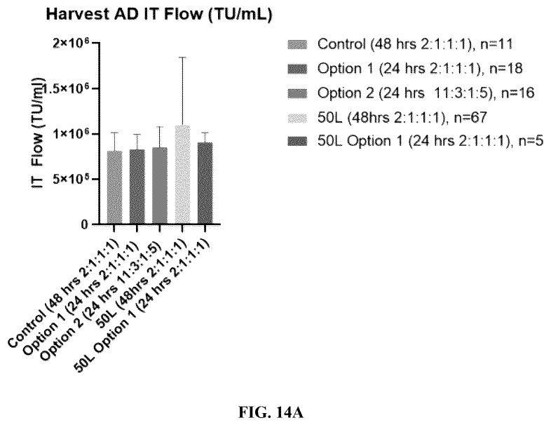

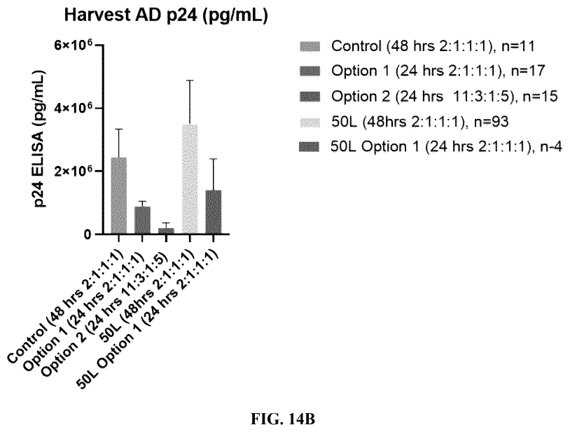

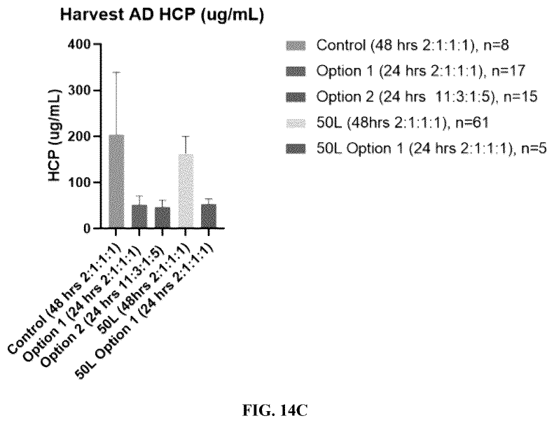

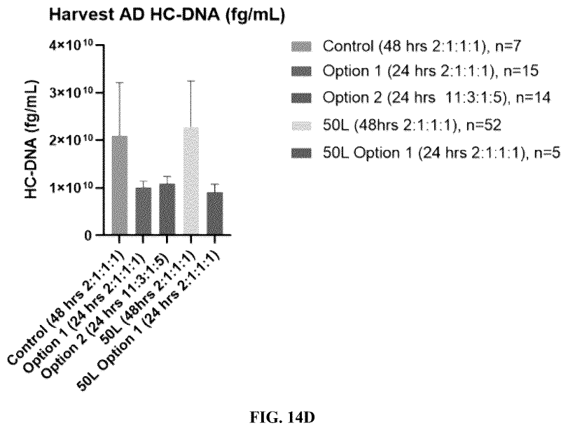

Both the infectious titers and impurity levels present at the time of harvest were evaluated. This involved sampling from the bioreactors at the designated harvest times, followed by analytical assays to quantify the infectious titers and impurity levels. The assessments provided a profile of the upstream harvest material. The three experimental conditions were evaluated, with the upstream results at harvest shown in FIGS. 14A-14D. These figures present specific results from both the 50 L Control condition and the 50 L Option 1 condition. The AD IT flow assay indicated that the 50 L bioreactor, when operated under Control conditions, produced a slightly higher titer than the smaller scales and the 50 L bioreactor operated under Option 1 conditions (FIG. 14A). The p24 levels were reduced for Option 1, Option 2, and the 50 L Option 1 condition compared to each of the Controls (FIG. 14B). The HCP and HC-DNA results show lower impurity levels under the 24-hour harvest conditions, even at the 50 L scale (FIGS. 14C-14D). These results suggest that the shorter harvest time effectively reduces impurity levels without compromising the infectious titer, even at the 11:3:1:5 vector ratio and the 50 L scale.

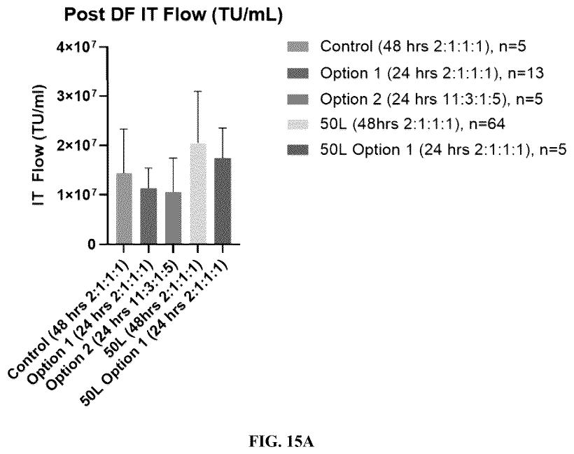

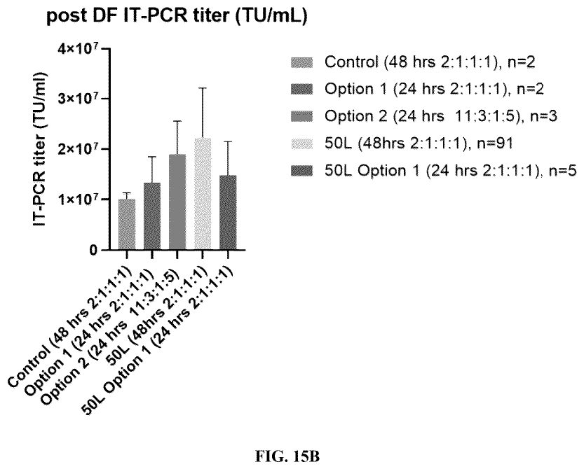

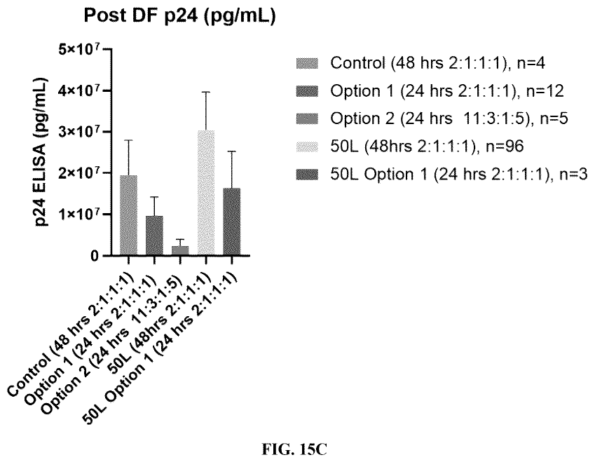

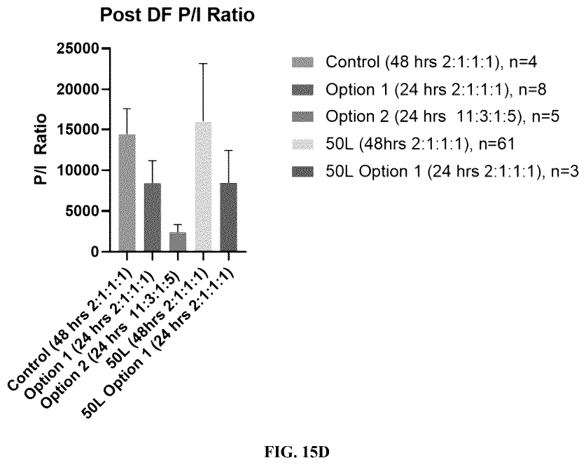

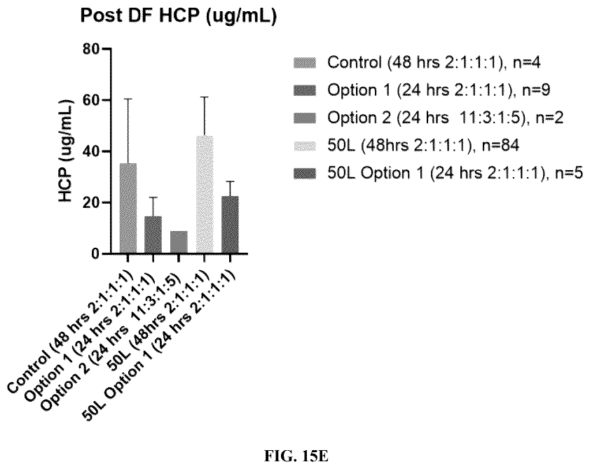

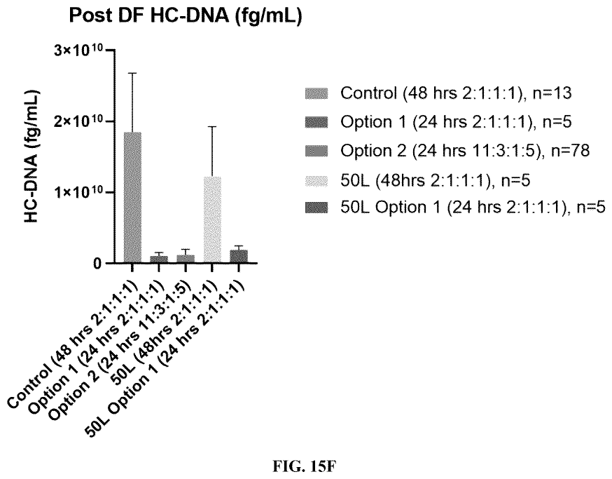

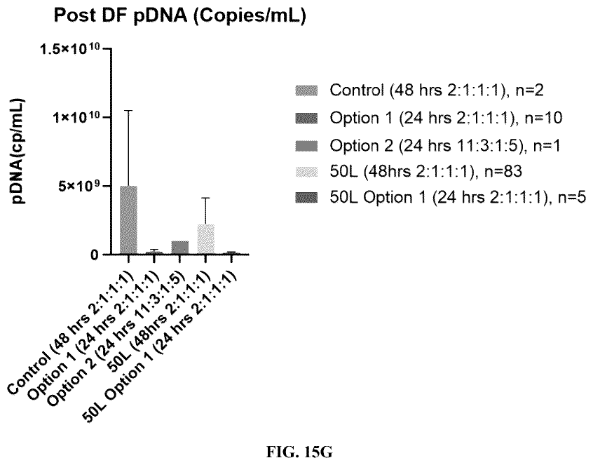

The downstream final drug substance LVV infectious titer was analyzed to evaluate the impact of different process conditions and bioreactor scales on the yield of infectious particles. The infectious titers, measured as IT-FLOW (FIG. 15A) and IT-PCR (FIG. 15B), were compared across the 2 L, 10 L, and 50 L bioreactor scales, with particular attention given to the results obtained from the 50 L bioreactors. The experimental conditions mirrored those in the upstream harvest experiment, with the Control and Option 1 conditions tested at all three scales, and Option 2 tested only at the 2 L and 10 L scales. Option 1 and Option 2 produced similar infectious titers (FIG. 15A), indicating that the reduced harvest time did not adversely affect the infectious yield. The 50 L bioreactor runs demonstrated a slightly higher IT-FLOW titer compared to the smaller scales (FIG. 15A), suggesting that the process scaled effectively to larger volumes. Furthermore, the 50 L Option 1 condition, with a 24-hour harvest time and a 2:1:1:1 vector ratio, yielded infectious titers comparable to the control but with the added benefit of a shorter production time (FIG. 15A). Overall, the data indicated that scaling up to a 50 L bioreactor did not compromise the infectious titer, with the 24-hour harvest time (Option 1) showing consistency across all tested scales. Maintaining or improving infectious titers at the 50 L scale highlights the potential for large-scale LVV production.

The downstream analysis of the final LVV product also included assessments of p24 levels (FIG. 15C) and the physical titer to infectious titer (P/I) ratio (FIG. 15D). The p24 protein, a component of the viral capsid, serves as a marker for the total number of viral particles. The P/I ratio provides insight into the proportion of functional (infectious) viral particles relative to the total number of particles, thereby offering a measure of the efficiency and quality of the LVV production process.

p24 levels and P/I ratios were compared across different bioreactor scales (2 L, 10 L, and 50 L) and process conditions using the same experimental conditions as previously described. As shown in FIG. 15C, p24 levels were reduced in both Option 1 (˜50% reduction) and Option 2 (˜87% reduction) compared to the Control. This reduction was consistent across the different scales, with the 50 L bioreactor under Option 1 conditions showing a decrease in p24 levels compared to the 50 L bioreactor under Control conditions. The reduced p24 levels indicate a lower presence of non-infectious particles, which is indicative of a higher quality vector preparation. FIG. 15D presents the P/I ratio data, showing similar trends. The P/I ratios for Options 1 and 2 were lower than those of the Control, further demonstrating the enhanced quality of the LVVs produced under these conditions. The 50 L bioreactor under Option 1 conditions exhibited P/I ratios comparable to those observed at smaller scales, indicating that the process scale-up did not adversely affect the ratio of infectious to non-infectious particles. In sum, both Option 1 and Option 2 demonstrated improvements over the Control condition, with these benefits maintained across the different bioreactor scales, including the 50 L scale. This suggests that these optimized conditions are robust and scalable.

The downstream analysis of the final LVV product also included an assessment of impurity levels, specifically host cell protein (HCP) (FIG. 15E), plasmid DNA (pDNA) (FIG. 15F), and host cell DNA (HC-DNA) (FIG. 15G). The same experimental conditions as previously described were applied. As depicted in FIG. 15E, HCP levels were reduced in both Option 1 and Option 2 compared to the Control. This reduction was consistent across all scales, with the 50 L bioreactor under Option 1 conditions showing a notable decrease in HCP levels compared to the 50 L Control. FIG. 15F depicts HC-DNA levels in the final LVV product, showing more than a one-log reduction in HC-DNA levels for both Option 1 and Option 2 compared to the Controls, including the Option 1 50 L set. This substantial reduction highlights the effectiveness of the 24-hour harvest time in minimizing the presence of host cell genetic material. The levels of pDNA were also significantly reduced under the 24-hour harvest conditions (FIG. 15G). Both Option 1 and Option 2 showed reductions in pDNA levels compared to the Control, with the 50 L bioreactor under Option 1 conditions again demonstrating particularly low levels of plasmid DNA. Overall, the data emphasize the improvements in impurity levels achieved through the optimized 24-hour harvest conditions. The reductions in HC-DNA, plasmid DNA, and HCP across all scales, particularly in the 50 L bioreactor, indicate that these conditions not only maintain high product yield and quality but also enhance the purity of the final LVV product.

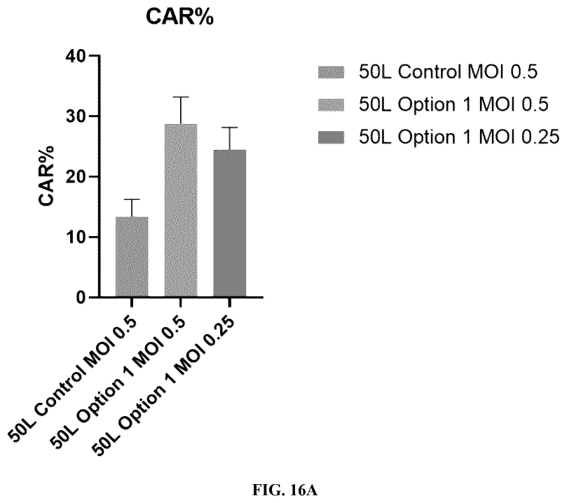

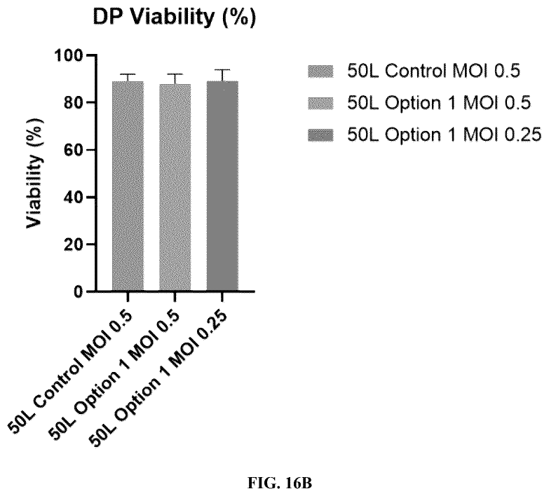

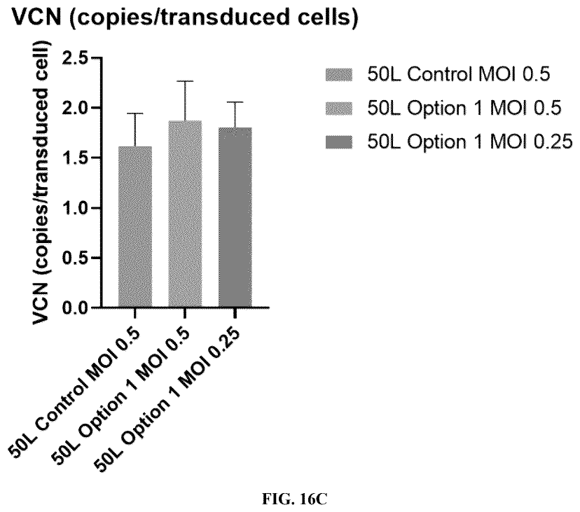

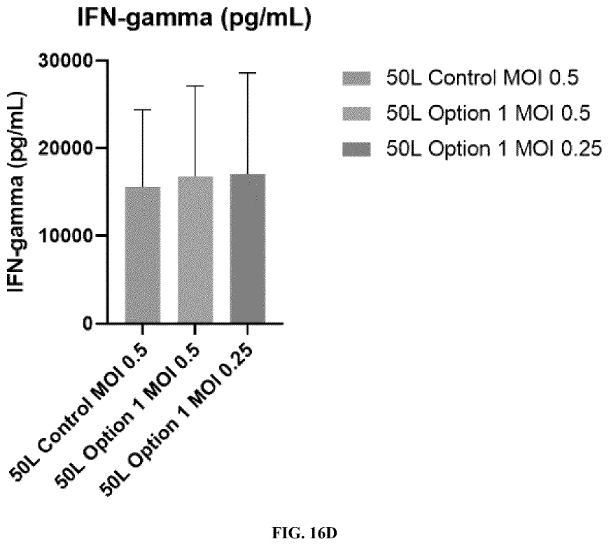

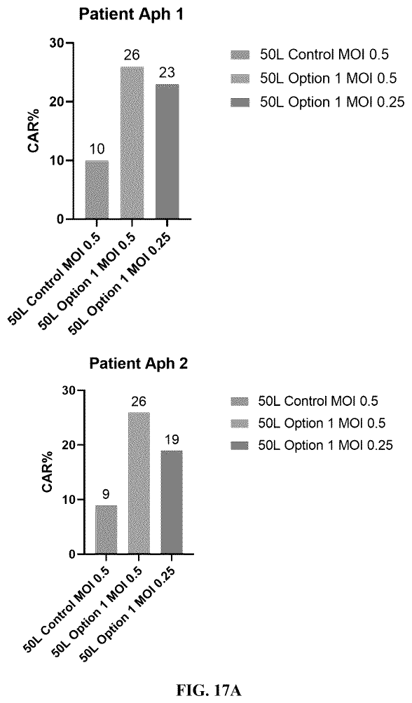

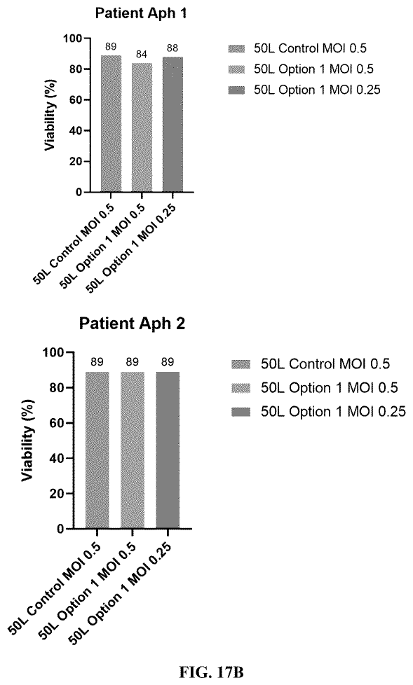

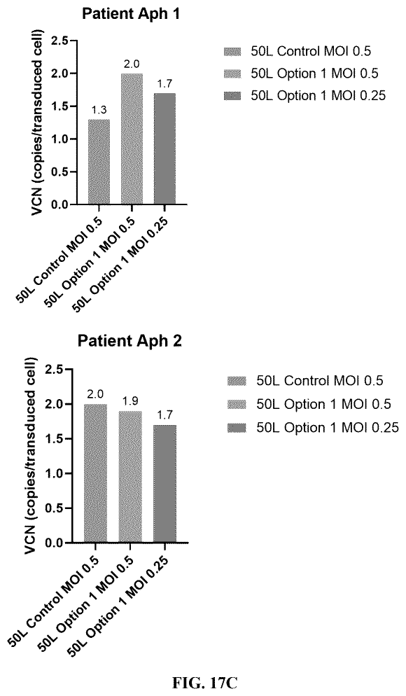

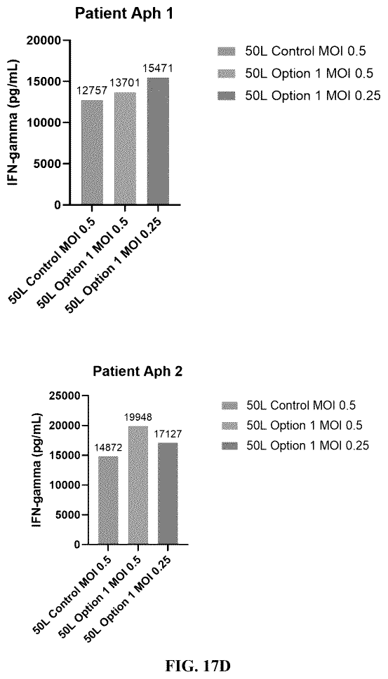

Next, transduction efficiency, viability, vector copy number, and potency were analyzed across different conditions at the 50 L scale. The transduction efficiency of CAR-T cells was evaluated, focusing on the percentage of CAR-positive cells. The Control group, which involved a 48-hour post-transfection harvest time with a 2:1:1:1 vector ratio, was compared with Option 1, which implemented a 24-hour post-transfection harvest time with the same vector ratio but at two different multiplicities of infection (MOI) levels: 0.5 and 0.25. As depicted in FIG. 16A and FIG. 17A, the 50 L scale runs in Option 1 demonstrated approximately a two-fold increase in transduction efficiency at both MOI 0.5 and 0.25, indicating a significant improvement over the Control. Additionally, patient-specific variations were observed, as shown in FIG. 17A, where the percentage of CAR-positive cells varied across individual patients.

The viability of the CAR-T drug product was assessed to determine the impact of the 24-hour harvest time (Option 1) compared to the 48-hour control at the 50 L scale. As shown in FIG. 16B and FIG. 17B, no significant differences in DP viability were observed between the Control and Option 1 conditions. The data suggest that shortening the harvest time to 24 hours did not adversely affect cell viability, as high levels of viability remained consistent with the Control condition. This consistency across the different MOI levels in Option 1 further supports the feasibility of the modified process without compromising product quality.

The vector copy number per transduced cell was also evaluated across the Control and Option 1 conditions. The results presented in FIG. 16C and FIG. 17C demonstrate that there were no significant differences in VCN between the Control and Option 1 conditions across various scales. This suggests that the 24-hour harvest time, along with the varied MOI levels in Option 1, did not compromise the genetic integrity of the CAR-T cells. The potency of the CAR-T drug product, measured by IFN-gamma levels, was also analyzed to assess the impact of the modified process conditions. FIG. 16D and FIG. 17D show that there was no observable impact on the potency of the CAR-T cells between the Control and Option 1 groups. The consistency in IFN-gamma levels across the different conditions further supports the efficacy of the 24-hour harvest time at both MOI levels.

The findings from the 50 L scale-up demonstrate the feasibility and potential advantages of implementing a 24-hour harvest time in conjunction with modified vector ratios in the production of CAR-T cell drug products. The 24-hour harvest time, particularly when paired with the optimized vector ratio of 2:1:1:1 at varied MOI levels, consistently produced comparable or superior outcomes in terms of transduction efficiency, viability, vector copy number, and potency. These results indicate that the process modifications did not compromise product quality and, in some cases, even enhanced key quality attributes. Overall, the successful application of these modified conditions at the 50 L scale supports their scalability and robustness.

1. A method of preparing a lentivirus, the method comprising:
transfecting host cells with a transfection composition, wherein the transfection composition comprises:
a first vector comprising a polynucleotide encoding a CAR,
a second vector, a third vector, and a fourth vector, and
a transfection media;
wherein the ratio of the first vector to the second vector to the third vector to the fourth vector is 2:1:1:1, 11:3:1:5, or 12:2:1:5,
culturing the transfected host cells to proliferate, and
harvesting the lentivirus, wherein the harvesting occurs about 24-hours after transfection.

2. A method of preparing a lentivirus, the method comprising:
transfecting host cells with a transfection composition, wherein the transfection composition comprises:
a first vector comprising a polynucleotide encoding a CAR,
a second vector, a third vector, and a fourth vector, and
a transfection media;
wherein the ratio of the first vector to the second vector to the third vector to the fourth vector is 11:3:1:5 or 12:2:1:5,
culturing the transfected host cells to proliferate, and
harvesting the lentivirus, wherein the harvesting occurs less than or equal to about 48-hours after transfection.

3. The method of claim 1, wherein the ratio is 2:1:1:1.

4. The method of claim 1, wherein the ratio is 11:3:1:5.

5. The method of claim 1, wherein the ratio is 12:2:1:5.

6. (canceled)

7. The method of claim 1, wherein the first vector, second vector, third vector, and fourth vector comprise lentiviral vectors.

8-10. (canceled)

11. The method of claim 1, wherein the second vector comprises a polynucleotide encoding a lentiviral envelope protein.

12. The method of claim 11, wherein the lentiviral envelope protein is vesicular stomatitis virus G (VSVG).

13. The method of claim 1, wherein the third vector comprises a polynucleotide encoding GAG and POL.

14. The method of claim 1, wherein the fourth vector comprises a polynucleotide encoding REV.

15. The method of claim 1, wherein the culturing occurs in a bioreactor.

16. The method of claim 15, wherein the bioreactor is a 2 L, 10 L or 50 L bioreactor.

17. The method of claim 1, wherein the host cells are HEK 293 cells.

18. The method of claim 1, wherein the CAR immunospecifically targets B-cell maturation antigen (BCMA).

19. A method of preparing a lentivirus, the method comprising:
transfecting host cells with a transfection composition, wherein the transfection composition comprises:
a first vector comprising a polynucleotide encoding a CAR that immunospecifically targets BCMA,
a second vector, a third vector, and a fourth vector, and
a transfection media;
wherein the ratio of the first vector to the second vector to the third vector to the fourth vector is 2:1:1:1, 11:3:1:5, or 12:2:1:5,
culturing the transfected host cells to proliferate, and
harvesting the lentivirus, wherein the harvesting occurs about 24-hours after transfection.

20. A method of preparing a lentivirus, the method comprising:
transfecting host cells with a transfection composition, wherein the transfection composition comprises:
a first vector comprising a polynucleotide encoding a CAR that immunospecifically targets BCMA,
a second vector, a third vector, and a fourth vector, and
a transfection media;
wherein the ratio of the first vector to the second vector to the third vector to the fourth vector is 11:3:1:5 or 12:2:1:5,
culturing the transfected host cells to proliferate, and
harvesting the lentivirus, wherein the harvesting occurs less than or equal to about 48-hours after transfection.

21. The method of claim 19, wherein the ratio is 2:1:1:1.

22. The method of claim 19, wherein the ratio is 11:3:1:5.

23. The method of claim 19, wherein the ratio is 12:2:1:5.

24. (canceled)

25. The method of claim 1, wherein the first vector, second vector, third vector, and fourth vector comprise lentiviral vectors.

26-28. (canceled)

29. The method of claim 1, wherein the second vector comprises a polynucleotide encoding a lentiviral envelope protein.

30. The method of claim 29, wherein the lentiviral envelope protein is vesicular stomatitis virus G (VSVG).

31. The method of claim 19, wherein the third vector comprises a polynucleotide encoding GAG and POL.

32. The method of claim 19, wherein the fourth vector comprises a polynucleotide encoding REV.

33. The method of claim 19, wherein the culturing occurs in a bioreactor.

34. The method of claim 33, wherein the bioreactor is a 2 L, 10 L or 50 L bioreactor.

35. The method of claim 19, wherein the host cells are HEK 293 cells.

36. The method of claim 19, wherein the first vector comprises SEQ ID NO: 1.

37. The method of claim 19, wherein the second vector comprises SEQ ID NO: 2.

38. The method of claim 19, wherein the third vector comprises SEQ ID NO: 3.

39. The method of claim 19, wherein the fourth vector comprises SEQ ID NO: 4.

40. The method of claim 19, wherein the CAR-T drug product is ciltacabtagene autolucel (cilta-cel).

41. A transfection composition, comprising:
a first vector comprising a polynucleotide encoding a CAR,
a second vector, a third vector, and a fourth vector, and
a transfection media;
wherein the ratio of the first vector to the second vector to the third vector to the fourth vector is 2:1:1:1, 11:3:1:5, or 12:2:1:5.

42.-53. (canceled)
